(19) United States
(12) Patent Application Publication
(10) Pub. No.: US 2021/0393811 A1
(43) Pub. Date: Dec. 23, 2021

(54) Imaging Agents and Methods of Use

(71) Applicant: Texas Heart Institute, Houston, TX (US)

(72) Inventors: Darren Woodside, Pearland, TX (US); Peter Vanderslice, Houston, TX (US); Robert Market, Pearland, TX (US); Ronald Biediger, Houston, TX (US); Richard Dixon, Houston, TX (US)

(21) Appl. No.: 17/279,504

(22) PCT Filed: Oct. 3, 2019
(86) PCT No.: PCT/US2019/054517

§ 371 Date: Mar. 24, 2021

Related U.S. Application Data
(60) Provisional application No. 62/741,747, filed on Oct. 5, 2018.

(51) Int. Cl. A61K 51/04; A61K 49/00

(52) U.S. Cl. CPC A61K 51/0459; A61K 49/0032; A61K 49/0002; A61K 49/0052

(57) ABSTRACT

A composition comprises a conjugate of the formula targeting component-linker-imaging component. In an embodiment, the targeting component is a VLA-4 antagonist. In an embodiment, the targeting component is a LFA-1 antagonist. In an embodiment, the linker includes chain of 2 to 20 atoms containing any combination of —CH₂—, —CH═CH—, —C(O)—, —NH—, —S—, —S(O)—, —O—, —C(O)O— or —S(O)₂—; or a polyethylene glycol chain, wherein said chain of 2-20 atoms or polyethylene glycol chain are attached to the targeting and imaging components through ether, amide, sulfonamide, urea, thiourea, or triazole functional groups. In an embodiment, the imaging component is a metal chelator complexed with a metal ion or isotope thereof.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD OF THE INVENTION

This disclosure relates generally to compositions including a targeting agent and methods of making and using. More specifically, this disclosure relates to chelators or dies attached to a targeting agent for use in medical application (e.g., for imaging and/or therapeutic purposes).

### BACKGROUND

A vulnerable plaque is a kind of atheromatous plaque—a collection of white blood cells (primarily macrophages) and lipids (including cholesterol) in the wall of an artery—that is particularly unstable and prone to produce sudden major problems such as a heart attack or stroke.

Inflammatory diseases include a vast array of disorders and conditions that are characterized by inflammation. Examples include allergy, asthma, autoimmune diseases, coeliac disease, glomerulonephritis, hepatitis, inflammatory bowel disease, reperfusion injury and transplant rejection.

An autoimmune disease is a condition in which your immune system mistakenly attacks your body. The immune system normally guards against germs like bacteria and viruses. When it senses these foreign invaders, it sends out an army of fighter cells to attack them. Normally, the immune system can tell the difference between foreign cells and your own cells. In an autoimmune disease, the immune system mistakes part of your body—like your joints or skin—as foreign. It releases proteins called autoantibodies that attack healthy cells. Some autoimmune diseases target only one organ. For example, Type 1 diabetes damages the pancreas. Other diseases, like lupus, affect the whole body.

There is a continuing need for developing imaging and therapeutic strategies to diagnose and treat such diseases.

### SUMMARY

Herein disclosed is a composition comprising a conjugate of the formula targeting component-linker-imaging component. In an embodiment, the targeting component is a VLA-4 antagonist. In an embodiment, the targeting component is a LFA-1 antagonist. In an embodiment, the linker includes a chain of 2 to 20 atoms containing any combination of —CH₂—, —CH═CH—, —C(O)—, —NH—, —S—, —S(O)—, —O—, —C(O)O— or —S(O)₂—; or a polyethylene glycol chain, wherein said linear chain of 2-20 atoms or polyethylene glycol chain are attached to the targeting and imaging components through ether, amide, sulfonamide, urea, thiourea, or triazole functional groups, which are included in the formula targeting component-linker-imaging component. Optionally, the linker can have an aryl or heterocyclic ring inserted in the chain. The linker may also be substituted with groups that improve physical characteristics, for example —SO₃H to increase water solubility.

In an embodiment, the imaging agent is a metal ion complexing agent. In an embodiment, the metal ion complexing agent is a DOTA (2-[4,7,10-tris(carboxymethyl)-1,4,7,10-tetrazacyclododec-1-yl]acetic acid) derivative, or a DTPA (diethylenetriamine pentaacetic acid) derivative, or a PCTA (3,6,9,15-Tetraazabicyclo[9.3.1] pentadeca-1(15),11,13-triene-3,6,9-triacetic acid) derivative. In an embodiment, the composition further comprises ions of Tm, Gd, Eu, Ho, Cu, Sn, Tc, In and radioisotopes thereof.

In an embodiment, the imaging component is a dye component. In an embodiment, the dye component comprises sulfo-Cy5, sulfo-Cy5.5, IR800CW, or Rhodamine 6G.

Also discussed herein is a method of using the composition in MRI and/or PET and/or NIRF imaging of vulnerable plaques in atherosclerosis; or MRI and/or PET and/or NIRF imaging of lung inflammation in acute lung injury; or MRI and/or PET and/or NIRF imaging of inflamed joints; or MRI and/or PET and/or NIRF imaging of tumors for diagnostic purposes, or purposes of validating therapeutic treatments; or MRI and/or PET and/or NIRF imaging of transplant rejection; or MRI and/or PET and/or NIRF imaging of aortic dissection/aneurysm. In an embodiment, inflamed joints comprise rheumatoid arthritis.

Further discussed is a method of using a composition comprising a conjugate of the formula “VLA-4 antagonist-linker-chelator” or “VLA-4 antagonist-linker-dye” or “LFA-1 antagonist-linker-chelator”or “LFA-1-antagonist-linker-dye” in anti-inflammatory or immunosuppressive drug delivery in atherosclerosis; or delivery of immunosuppressive therapeutics to immune cells to prevent acute or chronic transplant rejection; or delivery of immunosuppressive therapeutics to immune cells in autoimmune diseases; or delivery of therapeutic agents to tumors or malignant cells.

In an embodiment, the autoimmune diseases comprise multiple sclerosis or systemic lupus erythematosus. Where the terms VLA-4 antagonist-linker-chelator or VLA-4 antagonist-linker-dye are used, VLA-4 antagonist refers to a fragment capable of binding to the very late antigen-4 (VLA-4) integrin. Where the terms LFA-1 antagonist-linker-chelator or LFA-1 antagonist-linker-dye are used, LFA-1 antagonist refers to a fragment capable of binding lymphocyte function-associated antigen-1 (LFA-1) integrin.

The foregoing has outlined rather broadly the features and technical advantages of the invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### BRIEF DESCRIPTION OF THE DRAWINGS

For a more detailed description of the preferred embodiment of the present invention, reference will now be made to the accompanying drawings, wherein:

FIG. 1 shows conjugation of TBC3486. A. Structure of TBC3486. B. Structure of THI0510 (TBC3486 modified with conjugatable linker [TBC3486-conj] for functionalization with reactive dye and/or chelator reagents). C. TBC3486 and THI0510 inhibition of α4β1-K562 cell adhesion to the CS-1 sequence from fibronectin.

FIG. 2 shows THI375-based imaging compounds. A, B. Structure of THI520 and THI528. C. THI520 and THI0528 inhibition of α4β1-K562 cell adhesion to VCAM-1 (Mn++).

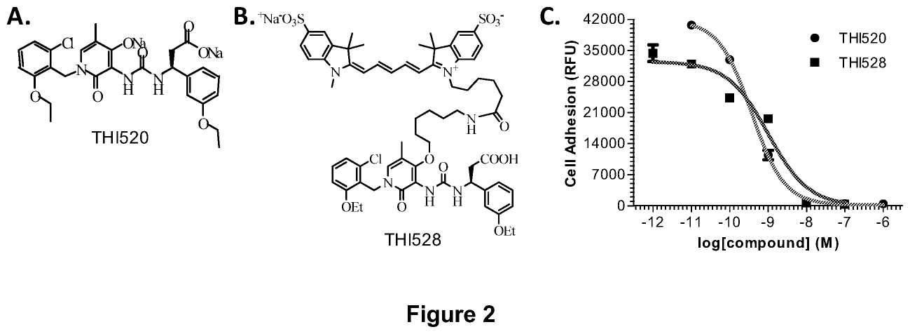

FIG. 3 shows flow cytometric analysis of THI528 binding to Jurkat(WT) and Jurkat(α4β1) cells. A. Saturable binding of THI528 with no detectable binding in presence of EDTA or to Jurkat(α4β1) cells. B. Individual histograms of 10 nM dose of THI528±EDTA.

FIG. 4 shows THI375-based imaging compounds. A. Conjugated THI375 analogue. B. THI375 analogue conjugated to the fluorescent dye sulfo-Cy5 (THI526). C. Activity of THI527, THI526, and TBC-4746 in K562-α4β1/CS-1 adhesion assays performed in Mn++. Calculated IC₅₀'s are shown in the table below the graph.

FIG. 5 illustrates structures of molecular probes targeting the integrin α4β1(5) and the β2 family (10b), including inactive controls.

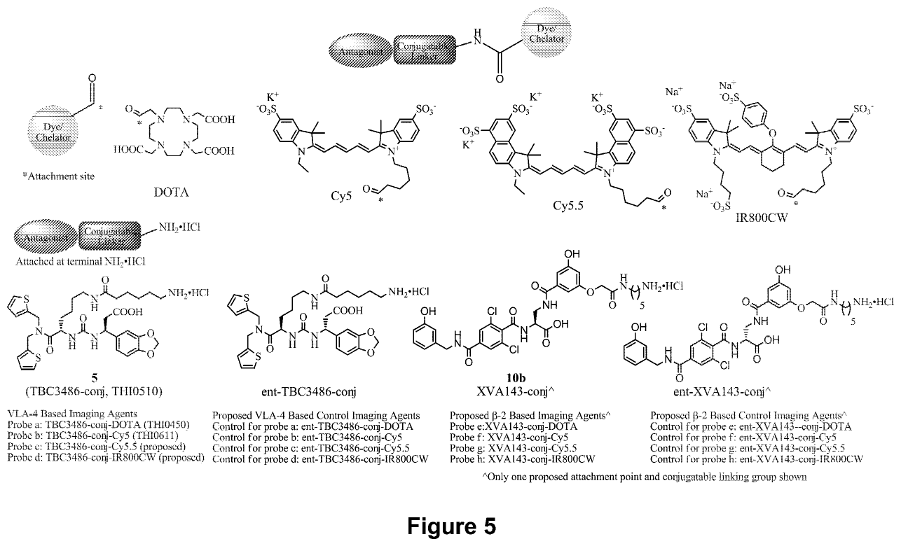

FIG. 6 illustrates how atoms of the linear chain of the linker are counted. In the example shown, the linker L¹ has the condensed formula —C₁₅—O₃—N₃—H₂₆—. The linker L¹ includes a linear chain of 19 atoms, numbered 1-19. 3 of the atoms of the chains, 9N, 10C, and 11C, together with their substituent —N═N—, form a heterocyclic ring, which is not substituted in this example.

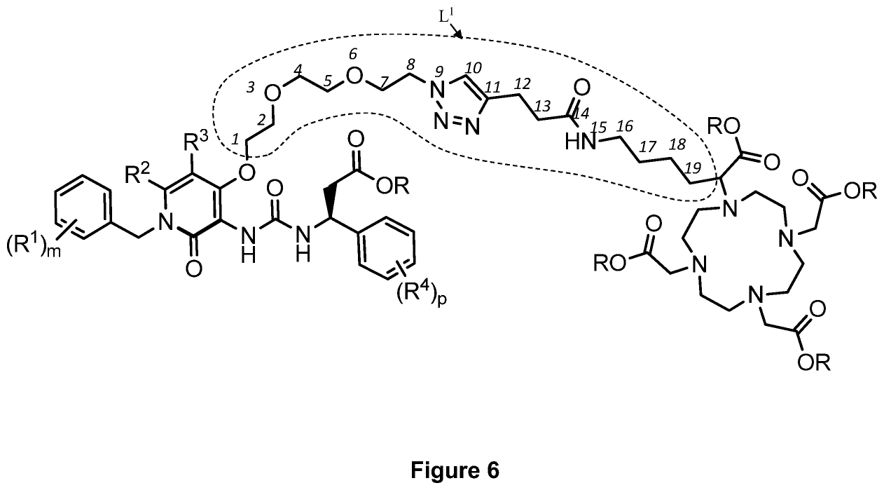

### DETAILED DESCRIPTION

Imaging vulnerable plaques, inflammatory diseases and autoimmunity are characterized by an accumulation of a variety of different types of cellular infiltrates. For example, about 50% of all the cellular components of atherosclerotic plaque are comprised of monocytes/macrophage and T lymphocytes. The integrin α4β1 (VLA-4) is highly expressed on monocytes and T lymphocytes. As a drug delivery tool, most hematologic malignancies involve cells expressing the integrin α4β1. The targeting agents of this disclosure may be used for locating tumors and metastases (in imaging modalities) and also for delivery of therapeutic drugs.

In an embodiment, modifications of integrin α4β1 and αLβ2 (LFA-1) antagonists with linker groups are made, which are amenable to modification with effector compounds.

In an embodiment, small molecule imaging agents are generated that specifically target the integrin α4β1, for use in intra-vital imaging, NIRF whole body imaging, PET imaging, and MRI. In an embodiment, such agents are used in drug delivery.

In an embodiment, regions within different core structures of integrin α4β1 antagonists are identified that can be conjugated with imaging agents and retain parent compound antagonist activity. Imaging agents include sulfo-Cy5 in two-photon intravital microscopy; sulfo-Cy5.5, IR800CW in NIRF whole body/organ imaging; Rhodamine 6G; metal chelators such as DOTA for chelating metals such as ions of Tm, Gd, Eu, Ho, Cu, Sn, Tc, In and radioisotopes thereof (e.g. ⁶⁴Cu) in MRI and PET imaging. Typical chelators include, but not limited to, DOTA derivatives (2-[4,7,10-tris(carboxymethyl)-1,4,7,10-tetrazacyclododec-1-yl]acetic acid), or a DTPA (diethylenetriamine pentaacetic acid) derivatives or derivatives based on PCTA 3,6,9,15-Tetraazabicyclo[9.3.1] pentadeca-1(15),11,13-triene-3,6,9-triacetic acid.

Such compounds (agents with conjugations) may be used in diagnosing inflammatory diseases and autoimmune diseases; tumor imaging and treatment; and detecting transplant rejection.

Table 1 shows the key features of the TBC3486-based imaging agents. Table 2 shows the features of THI520, a THI375-based analogue with increased α4β1 antagonist potency against both high and low affinity integrin, and with sufficient activity against murine integrin α4β1.

TABLE 1
Key features of the TBC3486-based imaging agents.

| | | Activity | | | | |
|---|---|---|---|---|---|---|
| Compound | | (IC₅₀) Cell | | | | General |
| Number | Structure | Type | Substrate | Cations | Species | Notes |
| THI516 (TBC3486- conj-Cy5) | 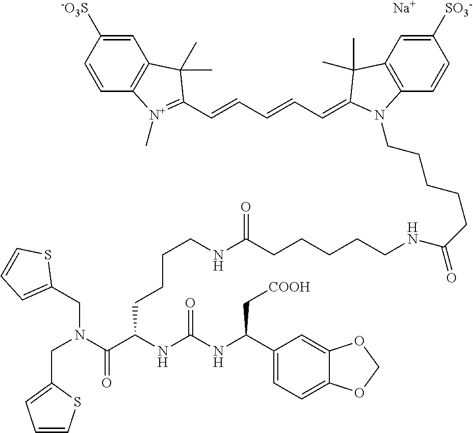 | 6.5 ± 3.5 nM (n = 4) K562(α4β1) 186.1 nM (n = 1) K562(α4β1) 6 ± 1 nM (n = 3) K562(α4β1) | CS-1 CS-1 Flow Cytometry | Mn Ca/Mg Mn | Human Human Human | Intravital microscopy showed high specific binding to what appears to be extracelfular matrix in the lung: There was no staining of murine lymphocytes in the lung. |
| THI517 (ent- TBC3486- conj-Cy5) | 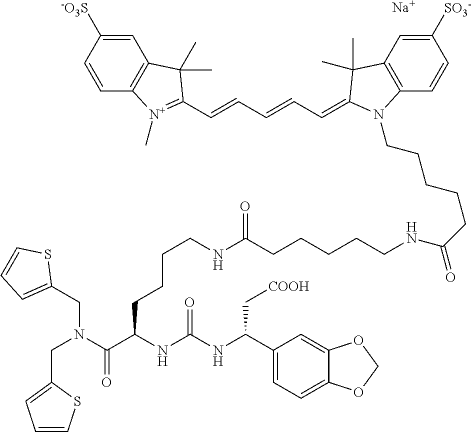 | 1.7 ± 1.0 uM (n = 3) K562(α4β1) >1 uM (n = 3) K562(α4β1) | CS-1 Flow Cytometry | Mn Mn | Human Human | Suitable α4β1 selectivity control for intravital imaging. |
| THI529 | 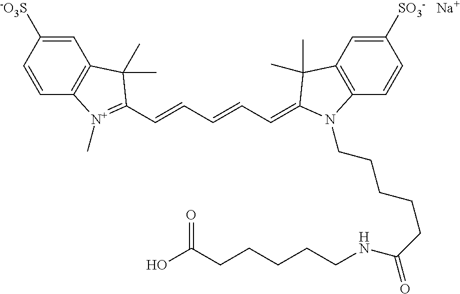 | No Activity (n = 3) K562(α4β1) | CS-1 | Mn | Human | Non- targeted control for intravital imaging. |

TABLE 2
Features of THI520, a THI375-based analogue with increased α4β1 antagonist potency against
both high and low affinity integrin, and with sufficient activity against murine integrin α4β1.

| Compound | | Activity (IC₅₀) | | | |
|---|---|---|---|---|---|
| Number | Structure | Cell Type | Substrate | Cations | Species |
| THI375 | 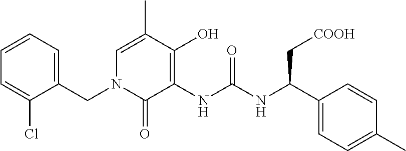 [SMILES: Cc1ccc([C@H](CC(=O)O)NC(=O)Nc2c(O)c(C)cn(Cc3ccccc3Cl)c2=O)cc1] | 2.2 ± 0.53 nM (n = 2) K562(α4β1) 1.4 ± 0.28 nM (n = 3) K562(α4β1) 810 ± 270 nM (n = 3) K562(α4β1) 127 ± 39 nM (n = 3) 70Z3 10,325 +/− 431 nM (n = 2) 70Z3 | CS-1 VCAM-1 VCAM-1 VCAM-1 VCAM-1 | Mn Mn Ca/Mg Mn Ca/Mg | Human Human Human Mouse Mouse |
| THI520 | 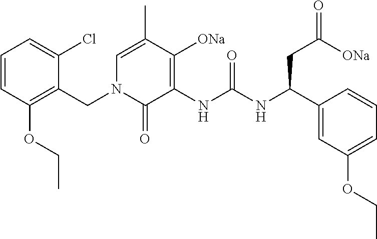 | 0.11 ± 0.04 nM (n = 2) K562(α4β1) 6.3 ± 3.1 nM (n = 3) K562(α4β1) 129 ± 42 nM (n = 3) 70Z3 0.75+/− 0.07 nM (n = 2) 70Z3 | CS-1 VCAM-1 VCAM-1 VCAM-1 | Mn Ca/Mg Ca/Mg Mn | Human Human Mouse Mouse |
| THI528 | 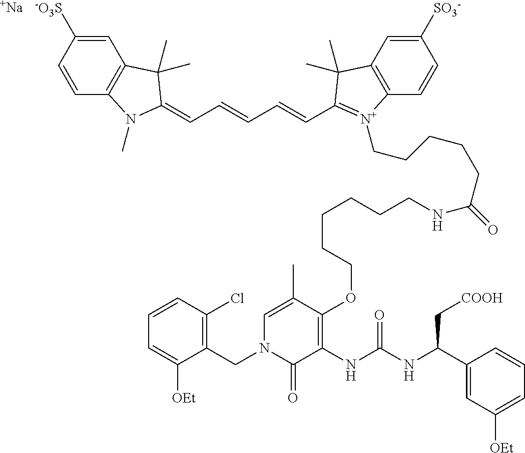 | 0.519 ± 0.097 nM (n = 3) K562(α4β1) 69 ± 16 pM (n = 3) Jurkat 202 ± 18 pM (n = 3) Jurkat 60 ± 11 pM (n = 3) 70Z3 | VCAM-1 Flow Cytometry Flow Cytometry Flow Cytometry | Mn Mn Ca/Mg Mn | Human Human Human Mouse |
| THI540 | 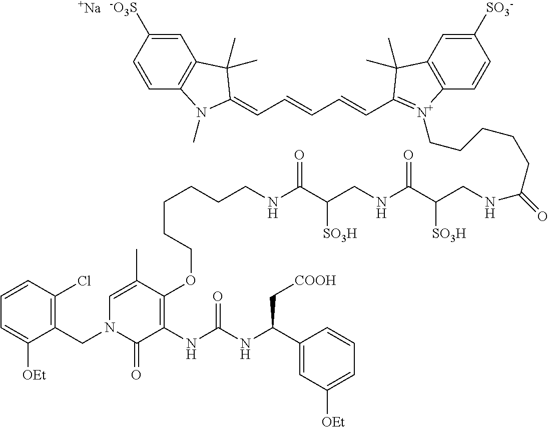 | 78 ± 37 pM (n = 3) Jurkat | Flow Cytometry | Mn | Human |

Use of integrin α4β1 or αLβ2 targeting ligand coupled to imaging agents to image autoimmune or inflammatory cell foci, for example atherosclerotic plaques, transplant rejection, joint inflammation in rheumatoid arthritis, lung inflammation in acute lung injury, or α4β1 or αLβ2 expressing tumors such as those found in lymphoma and multiple myeloma.

In various embodiments, the integrin targeting ligands of this disclosure are coupled to imaging agents to image autoimmune and/or inflammatory cell foci, or tumors, for use in:
MRI and/or PET and/or NIRF imaging of vulnerable plaques in atherosclerosis; or
MRI and/or PET imaging of lung inflammation in acute lung injury; or
MRI and/or PET imaging of inflamed joints such as in rheumatoid arthritis; or
MRI and/or PET imaging of tumors for diagnostic purposes, or purposes of validating therapeutic treatments; or
MRI and/or PET imaging of transplant rejection; or
MRI and/or PET imaging of aortic dissection/aneurysm.

In various embodiments, the integrin targeting ligands of this disclosure may be either formulated with therapeutics to target autoimmune or inflammatory cell foci, or cancer, for use in:
anti-inflammatory or immunosuppressive drug delivery in atherosclerosis or other inflammatory/autoimmune disorders; or
delivery of immunosuppressive therapeutics to immune cells to prevent acute or chronic transplant rejection; or
delivery of immunostimulatory therapeutics to immune cells to augment the immune response in diseases such as cancer or augment vaccines; or
delivery of immunosuppressive therapeutics to immune cells in autoimmune diseases like multiple sclerosis or systemic lupus erythematosus; or
delivery of therapeutic agents to tumors or malignant cells.

Various embodiments of this disclosure include pharmaceutically acceptable salts and carriers. Derivatives such as esters, carbamates, aminals, amides, optical isomers and pro-drugs are also contemplated.

### Definitions

The term “alkyl” as used herein, alone or in combination, refers to C₁-C₂₂ straight or branched, substituted or unsubstituted saturated chain radicals derived from saturated hydrocarbons by the removal of one hydrogen atom, unless the term alkyl is preceded by a Cₓ-C_{y} designation. Representative examples of alkyl groups include methyl, ethyl, n-propyl, iso-propyl, n-butyl, sec-butyl, iso-butyl, tert-butyl, and stearyl among others.

The term “alkenyl” as used herein, alone or in combination, refers to a substituted or unsubstituted straight-chain or substituted or unsubstituted branched-chain alkenyl radical containing from 2 to 22 carbon atoms. The term alkenyl as used herein can be taken to mean a chain containing one or more degrees of unsaturation. Examples of such radicals include, but are not limited to, ethenyl, E- and Z-pentenyl, decenyl, docosa-3,6,9,12,15,18-hexaenyl and the like.

The term “alkynyl” as used herein, alone or in combination, refers to a substituted or unsubstituted straight or substituted or unsubstituted branched chain alkynyl radical containing from 2 to 10 carbon atoms. Examples of such radicals include, but are not limited to ethynyl, propynyl, propargyl, butynyl, hexynyl, decynyl and the like.

The term “lower” modifying “alkyl”, “alkenyl”, “alkynyl” or “alkoxy” refers to a C₁-C₆ unit for a particular functionality. For example lower alkyl means C₁-C₆ alkyl.

The term “aliphatic acyl” as used herein, alone or in combination, refers to radicals of formula alkyl-C(O)—, alkenyl-C(O)— and alkynyl-C(O)— derived from an alkane-, alkene- or alkyncarboxylic acid, wherein the terms “alkyl”, “alkenyl” and “alkynyl” are as defined above. Examples of such aliphatic acyl radicals include, but are not limited to, acetyl, propionyl, butyryl, valeryl, 4-methylvaleryl, acryloyl, propiolyl and methylpropiolyl, among others.

The term “cycloalkyl” as used herein refers to an aliphatic ring system having 3 to 10 carbon atoms and 1 to 3 rings, including, but not limited to cyclopropyl, cyclopentyl, cyclohexyl, norbornyl, and adamantyl among others. Cycloalkyl groups can be unsubstituted or substituted with one, two or three substituents independently selected from lower alkyl, haloalkyl, alkoxy, thioalkoxy, amino, alkylamino, dialkylamino, hydroxy, halo, mercapto, nitro, carboxaldehyde, carboxy, alkoxycarbonyl and carboxamide.

Substituted “cycloalkyl” includes cis or trans forms. Furthermore, the substituents may either be in endo or exo positions in the bridged bicyclic systems.

The term “cycloalkenyl” as used herein alone or in combination refers to a cyclic carbocycle containing from 4 to 8 carbon atoms and one or more double bonds. Examples of such cycloalkenyl radicals include, but are not limited to, cyclopentenyl, cyclohexenyl, cyclopentadienyl and the like.

The term “cycloalkylalkyl” as used herein refers to a cycloalkyl group appended to a lower alkyl radical, including, but not limited to cyclohexylmethyl.

The term “halo” or “halogen” as used herein refers to I, Br, Cl or F.

The term “haloalkyl” as used herein refers to a lower alkyl radical, to which is appended at least one halogen substituent, for example chloromethyl, fluoroethyl, trifluoromethyl and pentafluoroethyl among others.

The term “alkoxy” as used herein, alone or in combination, refers to an alkyl ether radical, wherein the term “alkyl” is as defined above. Examples of suitable alkyl ether radicals include, but are not limited to, methoxy, ethoxy, n-propoxy, iso-propoxy, n-butoxy, iso-butoxy, sec-butoxy, tert-butoxy and the like.

The term “alkoxyalkyl” as used herein, refers to R^{Y}—O—R^{z}, wherein R^{Y} is lower alkyl as defined above, and R^{z} is alkylene (—(CH₂)_{w}—) wherein “w” is an integer of from one to six.

Representative examples include methoxymethyl, methoxyethyl, and ethoxyethyl among others.

The term “alkenoxy” as used herein, alone or in combination, refers to a radical of formula alkenyl-O—, provided that the radical is not an enol ether, wherein the term “alkenyl” is as defined above. Examples of suitable alkenoxy radicals include, but are not limited to, allyloxy, E- and Z-but-2-en-1-yloxy and the like.

The term “alkynoxy” as used herein, alone or in combination, refers to a radical of formula alkynyl-O—, provided that the radical is not an -ynol ether. Examples of suitable alkynoxy radicals include, but are not limited to, propargyloxy, 2-butynyloxy and the like.

The term “carboxy” as used herein refers to C(O)OH.

The term “thioalkoxy” refers to a thioether radical of formula alkyl-S—, wherein “alkyl” is as defined above.

The term “sulfonamido” as used herein refers to —SO₂NH₂.

The term “carboxaldehyde” as used herein refers to —C(O)R wherein R is hydrogen.

The terms “carboxamide” or “amide” as used herein refer to C(O)NR^{a}R^{b} wherein R^{a} and R^{b} are each independently hydrogen, alkyl or any other suitable substituent.

The term “alkoxyalkoxy” as used herein refers to R^{c}O—R^{d}O— wherein R^{c} is lower alkyl as defined above and R^{d} is alkylene wherein alkylene is —(CH₂)ₙ— wherein n is an integer from 1 to 6. Representative examples of alkoxyalkoxy groups include methoxymethoxy, ethoxymethoxy, t-butoxymethoxy among others.

The term “alkylamino” as used herein refers to R^{e}NH— wherein R^{e} is a lower alkyl group, for example, ethylamino, butylamino, among others.

The term “alkenylamino” as used herein, alone or in combination, refers to a radical of formula alkenyl-NH— or (alkenyl)₂N—, wherein the term “alkenyl” is as defined above, provided that the radical is not an enamine. An example of such alkenylamino radical is the allylamino radical.

The term “alkynylamino” as used herein, alone or in combination, refers to a radical of formula alkynyl-NH— or (alkynyl)₂N— wherein the term “alkynyl” is as defined above, provided that the radical is not an -ynamine. An example of such alkynylamino radicals as used herein is the propargyl amino radical HC≡C—CH₂NH—.

The term “dialkylamino” as used herein refers to (R^{f})(R^{g})N— wherein R^{f} and R^{g} are independently selected from lower alkyl, for example diethylamino, and methyl propylamino, among others.

The term “alkoxycarbonyl” as used herein refers to an alkoxyl group as previously defined appended to the parent molecular moiety through a carbonyl group. Examples of alkoxycarbonyl include methoxycarbonyl, ethoxycarbonyl, and isopropoxycarbonyl among others.

The term “aryl” or “aromatic” as used herein alone or in combination refers to a substituted or unsubstituted carbocyclic aromatic group having about 6 to 12 carbon atoms such as phenyl, naphthyl, indenyl, indanyl, azulenyl, fluorenyl and anthracenyl; or a heterocyclic aromatic group containing at least one endocyclic N, O or S atom such as furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, indolizinyl, indolyl, isoindolyl, 3H-indolyl, indolinyl, benzo [b] furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, pyrazolo[1,5-c]triazinyl and the like. “Aralkyl” and “alkylaryl” employ the term “alkyl” as defined above. Rings may be multiply substituted.

The term “aralkyl” as used herein, alone or in combination, refers to an aryl substituted alkyl radical, wherein the terms “alkyl” and “aryl” are as defined above. Examples of suitable aralkyl radicals include, but are not limited to, phenylmethyl, phenethyl, phenylhexyl, diphenylmethyl, pyridylmethyl, tetrazolyl methyl, furylmethyl, imidazolyl methyl, indolylmethyl, thienylpropyl and the like.

The term “aralkenyl” as used herein, alone or in combination, refers to an aryl substituted alkenyl radical, wherein the terms “aryl” and “alkenyl” are as defined above.

The term “arylamino” as used herein, alone or in combination, refers to a radical of formula aryl-NH—, wherein “aryl” is as defined above. Examples of arylamino radicals include, but are not limited to, phenylamino (anilido), naphthyl amino, 2-, 3-, and 4-pyridylamino and the like.

The term “benzyl” as used herein refers to C₆H₅—CH₂—.

The term “biaryl” as used herein, alone or in combination, refers to a radical of formula aryl-aryl, wherein the term “aryl” is as defined above.

The term “thioaryl” as used herein, alone or in combination, refers to a radical of formula aryl-S— wherein the term “aryl” is as defined above. An example of a thioaryl radical is the phenylthio radical.

The term “aroyl” as used herein, alone or in combination, refers to a radical of formula aryl-CO—, wherein the term “aryl” is as defined above. Examples of suitable aromatic acyl radicals include, but are not limited to, benzoyl, 4-halobenzoyl, 4-carboxybenzoyl, naphthoyl, pyridylcarbonyl and the like.

The term “heterocyclyl” as used herein, alone or in combination, refers to a non-aromatic 3- to 10-membered ring containing at least one endocyclic N, O, or S atom. The heterocycle may be optionally aryl-fused. The heterocycle may also optionally be substituted with at least one substituent which is independently selected from the group consisting of hydrogen, halogen, hydroxyl, amino, nitro, trifluoromethyl, trifluoromethoxy, alkyl, aralkyl, alkenyl, alkynyl, aryl, cyano, carboxy, carboalkoxy, carboxyalkyl, oxo, arylsulfonyl and aralkylaminocarbonyl among others.

The term “alkylheterocyclyl” as used herein refers to an alkyl group as previously defined appended to the parent molecular moiety through a heterocyclyl group, including but not limited to 4-methyl piperazin-1-yl.

The term “heterocyclylalkyl” as used herein refers to a heterocyclyl group as previously defined appended to the parent molecular moiety through an alkyl group, including but not limited to 2-(1-piperidinyl)ethyl.

The term “heterocycloyl” as used herein refers to radicals of the formula heterocyclyl-C(O)—, wherein the term “hetercyclyl” is as defined above.

The term “aminal” as used herein refers to a radical of the structure R^{h}C(NRⁱR^{j})(NR^{k}R^{l})— wherein R^{h}, Rⁱ, R^{j}, R^{k} and R^{l} are each independently hydrogen, alkyl or any other suitable substituent.

The term “ester” as used herein refers to —CO₂R^{m}, wherein R^{m} is alkyl or any other suitable substituent.

The term “carbamate” as used herein refers to compounds based on carbamic acid —NXC(O)OR, wherein for example, X is hydrogen, alkyl, aryl or aralkyl and independently R is alkyl, aryl or aralkyl.

The term “radical” as used herein refers to an atom or group of atoms derived from a neutral molecule by the removal of one or more atoms, where the radical is attached to another radical by means of a covalent bond.

The term “optical isomers” as used herein refers to compounds which differ only in the stereochemistry of at least one atom, including enantiomers, diastereomers and racemates.

Use of the above terms is meant to encompass substituted and unsubstituted moieties. Substitution may be by one or more groups such as alcohols, ethers, esters, amides, sulfones, sulfides, hydroxyl, nitro, cyano, carboxy, amines, heteroatoms, lower alkyl, lower alkoxy, lower alkoxycarbonyl, alkoxyalkoxy, acyloxy, halogens, trifluoromethoxy, trifluoromethyl, alkyl, aralkyl, alkenyl, alkynyl, aryl, cyano, carboxy, carboxyalkyl, cycloalkyl, cycloalkylalkyl, heterocyclyl, alkylheterocyclyl, heterocyclylalkyl, oxo, arylsulfonyl and aralkylaminocarbo-nyl or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)₂—. Rings may be substituted multiple times.

The terms “electron-withdrawing” or “electron-donating” refer to the ability of a substituent to withdraw or donate electrons relative to that of hydrogen if hydrogen occupied the same position in the molecule. These terms are well-understood by one skilled in the art and are discussed in Advanced Organic Chemistry by J. March, 1985, pp. 16-18, incorporated herein by reference. Electron withdrawing groups include halo, nitro, carboxy, lower alkenyl, lower alkynyl, carboxaldehyde, carboxyamido, aryl, quaternary ammonium, trifluoromethyl, sulfonyl and aryl lower alkanoyl among others. Electron donating groups include such groups as hydroxy, lower alkyl, amino, lower alkylamino, di(lower alkyl)amino, aryloxy, mercapto, lower alkylthio, lower alkylmercapto, and disulfide among others. One skilled in the art will appreciate that the aforesaid substituents may have electron donating or electron with-drawing properties under different chemical conditions. Moreover, the present invention contemplates any combi-nation of substituents selected from the above-identified groups.

The most preferred electron donating or electron with-drawing substituents are halo, nitro, alkanoyl, carboxaldehyde, arylalkanoyl, aryloxy, carboxyl, carboxamide, cyano, sulfonyl, sulfoxide, heterocyclyl, guanidine, quaternary ammonium, lower alkenyl, lower alkynyl, sulfonium salts, hydroxy, lower alkoxy, lower alkyl, amino, lower alkylamino, di(lower alkyl)amino, amine lower alkyl mercapto, mercaptoalkyl, alkylthio, carboxy lower alkyl, arylalkoxy, alkanoylamino, alkanoyl (lower alkyl)amino, lower alkylsufonylamino, arylsulfonylamino, alkylsulfonyl(lower alkyl)amino, arylsulfonyl(lower alkyl) amino, lower alkylcarboxamide, di(lower alkyl) carboxamide, sulfonamide, lower alkyl sulfonamide, di(lower alkyl)sulfonamide, lower alkylsulfonyl, arylsulfonyl and alkyldithio.

Pharmaceutically acceptable salts are well known in the art. For example, S. M. Berge, et al., describe pharmaceutically acceptable salts in detail in J. Pharmaceutical Sciences, 66: 1-19 (1977). The salts can be prepared in situ during the final isolation and purification of the compounds taught herein, or separately by reacting a free base or free acid function with a suitable reagent, as described generally below. For example, a free base function can be reacted with a suitable acid. Furthermore, where the compounds taught herein carry an acidic moiety, suitable pharmaceutically acceptable salts thereof may, without limiting the scope of the invention, include metal salts such as alkali metal salts, e.g., sodium or potassium salts; and alkaline earth metal salts, e.g., calcium or magnesium salts.

### Abbreviations

ACN Acetonitrile
Boc, BOC tert-Butoxycarbonyl
Boc-Dap-OH 3-amino-2-((tert-butoxycarbonyl)amino)propanoic acid
CDI 1,1′-Carbonyl diimidazole
CS-1 Connecting segment-1
Cy Cyanine
DCC Dicyclohexylcarbodiimide
DCM Dichloromethane
DIPEA N,N-Disopropylethylamine
DMF N,N-Dimethylformamide
DOTA (2-[4,7,10-tris(carboxymethyl)-1,4,7,10-tetrazacyclododec-1-yl]acetic acid)
EDCI N-(3-Dimethylaminopropyl)-N′-ethylcarbodimide hydrochloride
Fmoc 2-((9H-Fluoren-9-yl)methoxy)carbonyl
HBTU O-(Benzotriazol-1-yl)-N,N,N′,N′-tetramethyluronium hexafluorophosphate
HPLC High performance liquid chromatography
IC₅₀ Half maximal inhibitory concentration
IR800CW 800 nm channel near-infrared dye
Kd Dissociation constant
LCMS Liquid Chromatography-Mass Spectrometry
LFA-1 (αLβ2) Lymphocyte function-associated antigen-1
MRI Magnetic resonance imaging
NHS N-Hydroxysuccinimide
NIRF Near infrared fluorescence 3,6,9,15-Tetraazabicyclo[9.3.1] pentadeca-1(15),11,13-triene-3,6,9-triacetic
PCTA acid
PET Positron-emission tomography
Su N-succinimidyl
TFA, Tfa Trifluoroacetic acid
TSTU N,N,N′,N′-tetramethyl-O—(N-succinimidyl)uronium tetrafluorborate
VCAM-1 Vascular cell adhesion molecule 1
VLA-4(α4β1) Very late antigen 4

### EXAMPLES

### Affinity of VLA-4 Antagonists

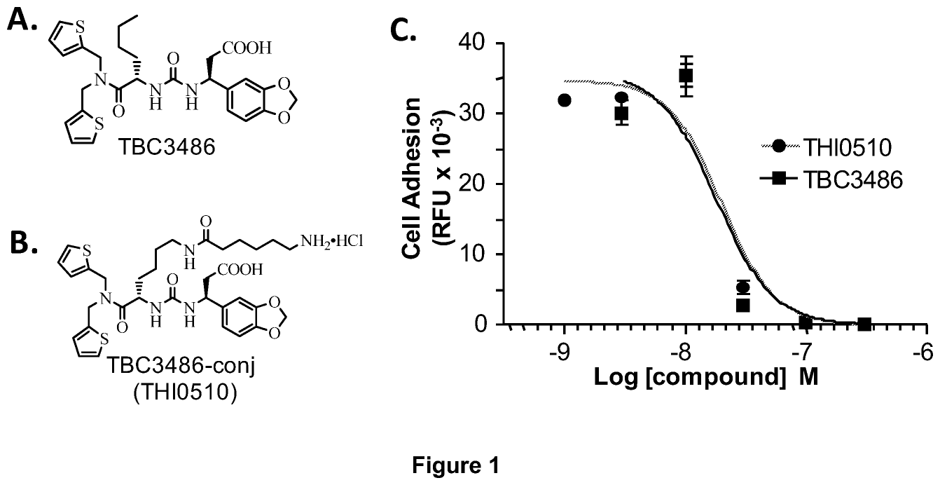

First attempts to generate an integrin α4β1 imaging agent took advantage of the TBC3486 core scaffold. Modifications to the core scaffold were generated to determine an appropriate site to which imaging conjugates could be attached (FIG. 1). A conjugation site was identified (THI510) that did not significantly influence antagonist potency (FIG. 1). Next, imaging agents were attached to this conjugation site and their activities tested in cell adhesion assays. Overall, attachment of the dye sulfo-Cy5, sulfo-Cy5.5, IR800CW, or the chelating agent like DOTA, did not significantly affect TBC3486 potency (not shown).

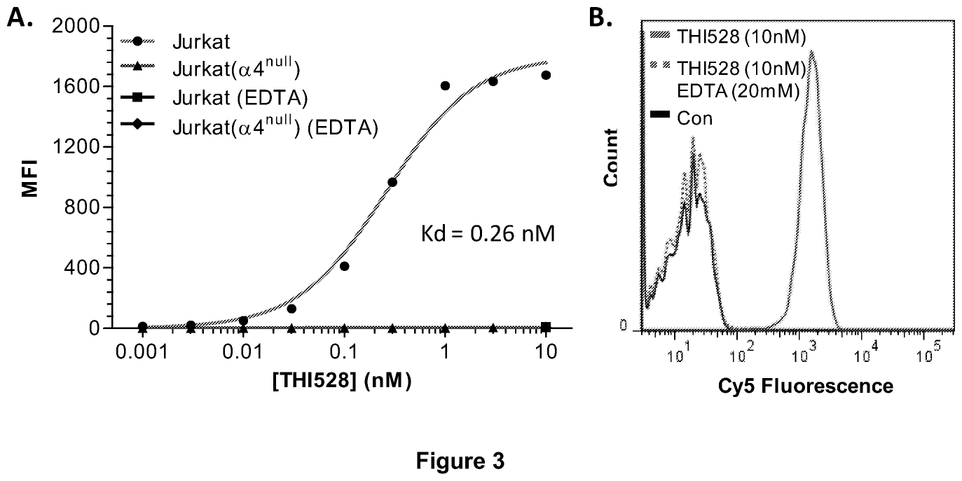

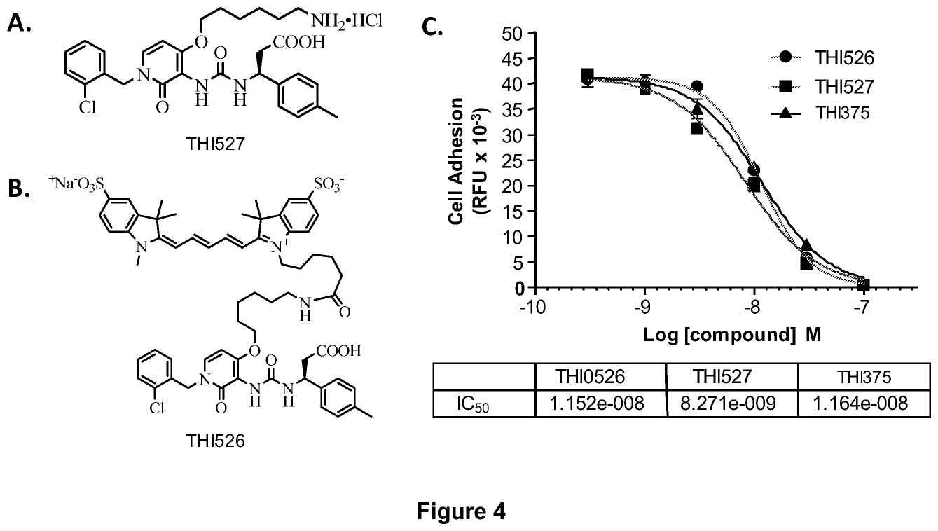

THI520 has an IC₅₀ of 6.3±3.1 nM in low affinity α4β1 adhesion assays, and pM activity in high affinity assays. The core of this structure is different than that of TBC3486 (FIG. 3A). THI520 can be conjugated to sulfo-Cy5 (THI528, FIG. 3B), with no apparent loss in antagonist activity (FIG. 3C). THI528 was tested for binding to integrin α4β1by flow cytometry (FIG. 4). Jurkat cells that express the integrin α4β1 (Jurkat(α4β1)), or mutagenized Jurkat cells that no longer express the integrin α4β1 (Jurkat(α4^{null})), where incubated with increasing concentrations of THI528. Specific and saturable binding was observed (FIG. 4), with an apparent Kd of 0.26 nM (n=1).

An example of this is THI520 (See KEY MOLECULES, Table 2). It has an IC₅₀ of 6.3±3.1 nM in low affinity α4β1 adhesion assays. The core of this structure is based on THI375. The THI375 core can be modified with imaging agents that do not significantly affect α4β1 binding.

### Synthesis of VLA-4 Antagonist Intermediates

VLA-4 antagonist based imaging agent can be synthetized from the intermediates shown in examples 1-3. Linking groups can vary depending on the chemistry employed to append the chelators and the desired changes to physical characteristics of the final conjugate.

### Example 1

The VLA-4 antagonists may be synthesized according to U.S. Pat. No. 6,723,711, incorporated herein by reference. To produce the general structure of a common amine intermediate. The intermediate was produced from the azide by triphenylphosphine/water reduction. Stereoisomers may alternatively be synthetized.

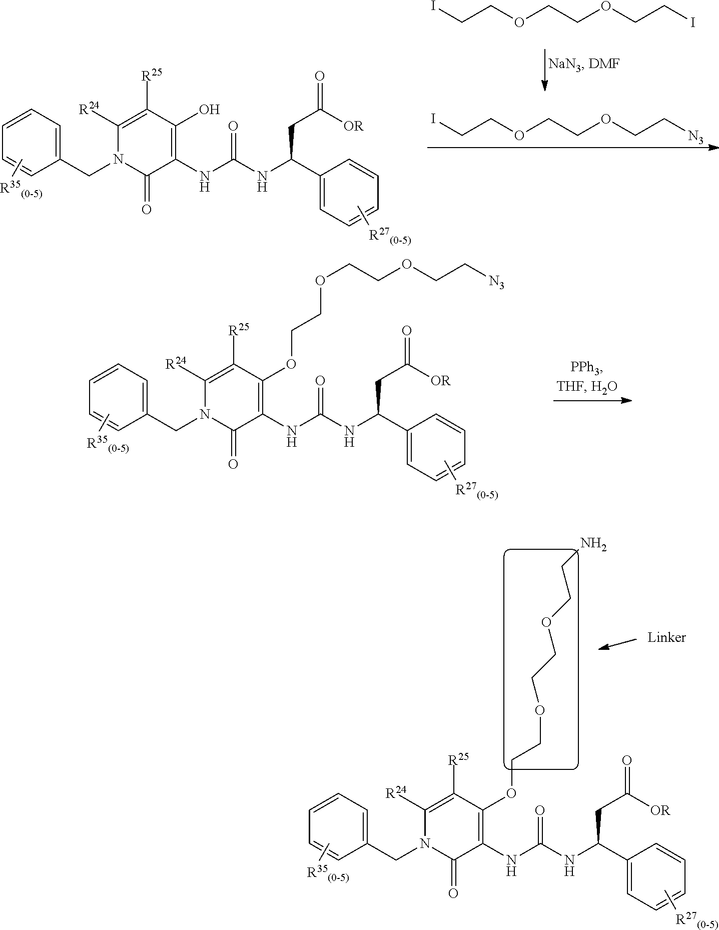

Note that R is a suitable alkyl protecting group or H and R²⁴, R²⁵, R²⁷ and R³⁵ are as defined in the same patent and are independently selected from H or groups listed therein. The linker includes a linear chain of atoms formed from any combination of the groups —CH₂—, —CH═CH—, —C(O)—, —NH—, —S—, —S(O)—, —O—, —C(O)O—, —S(O)₂—, that may be substituted or unsubstituted; it should be understood by one skilled in the art to be exemplary and may be as short as two atoms (e.g. —CH₂—CH₂—) or as long as 20 atoms, any of which may also be all carbon or short chained polyethylene glycol units. Optionally, the linker can have an aryl or heterocyclic ring inserted in the chain. Linkers may be modified to adjust physical properties as needed. For example, the insertion of one or more 2-sulfo-beta-alanine groups will increase hydrophilicity.

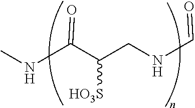

### Example 2

The azide below is a precursor to this amine and could be used to prepare VLA-4 targeted metal ion chelators by Click chemistry.

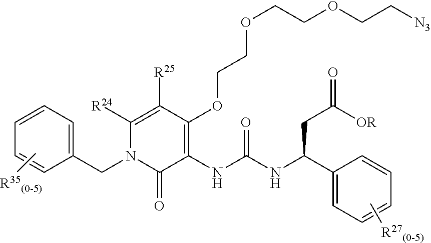

The functionality of the terminal amine can optionally be modified to a carboxylic acid group that can subsequently be activated with reagents such as TSTU and treated with amines to prepare amide linkages to either dyes or metal chelators such as DOTA. Any of the amine functionalized chelators below may be used with this reagent to prepare amide linked VLA-4 targeting chelators.

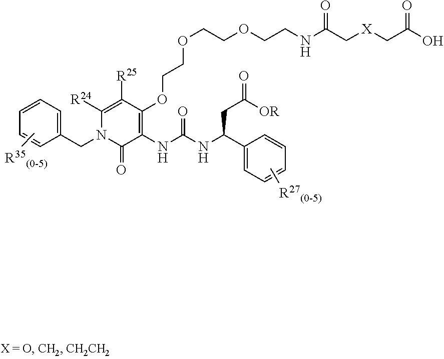

Based on the intermediate structure, a variety of metal chelators can be envisioned for imaging. The chelators chosen can coordinate metals with varying oxidative states. Stereoisomers may alternatively be synthetized.

### Example 3

Intermediates to prepare VLA-4 antagonist conjugates based on TBC3486 may be synthesized according to the methods contained in U.S. Pat. No. 6,194,448, which is incorporated herein by reference, to arrive at similarly based amine starting materials.

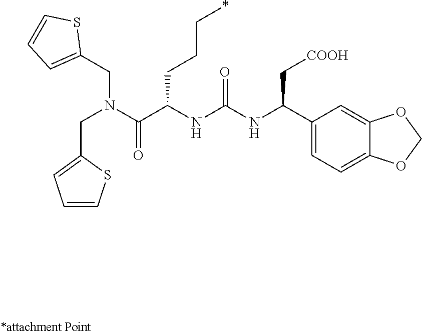 [SMILES: *CCC[C@H](NC(=O)N[C@@H](CC(=O)O)c1ccc2c(c1)OCO2)C(=O)N(Cc1cccs1)Cc1cccs1]

Stereoisomers may alternatively be synthetized.

### VLA-4 Antagonist-Linker-Chelator Conjugates

DOTA based VLA-4 antagonist conjugates may be produced according to standard methods for making amides, ureas, and thioureas. Other methods, such as Click chemistry, may also be employed to attach chelators to the VLA-4 targeting components described herein.

VLA-4 antagonist conjugates with chelators containing three (tridentate), four (tetradentate), or five (pentadentate) acidic coordination sites have been synthesized by the methods in the following examples 4-14. Stereoisomers may alternatively be synthetized.

### Example 4

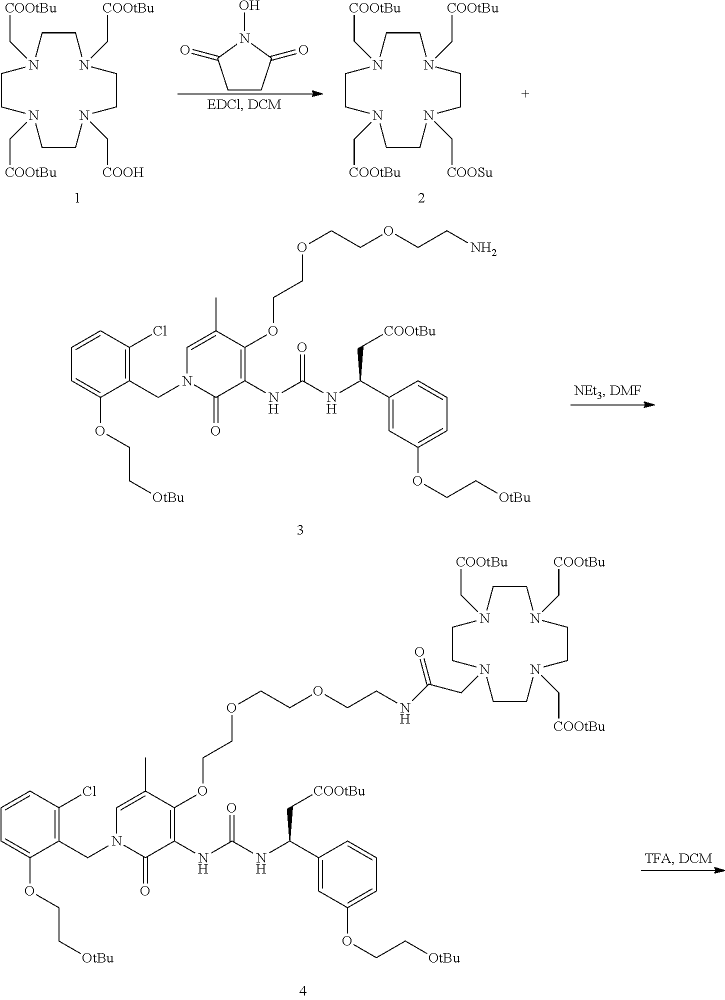

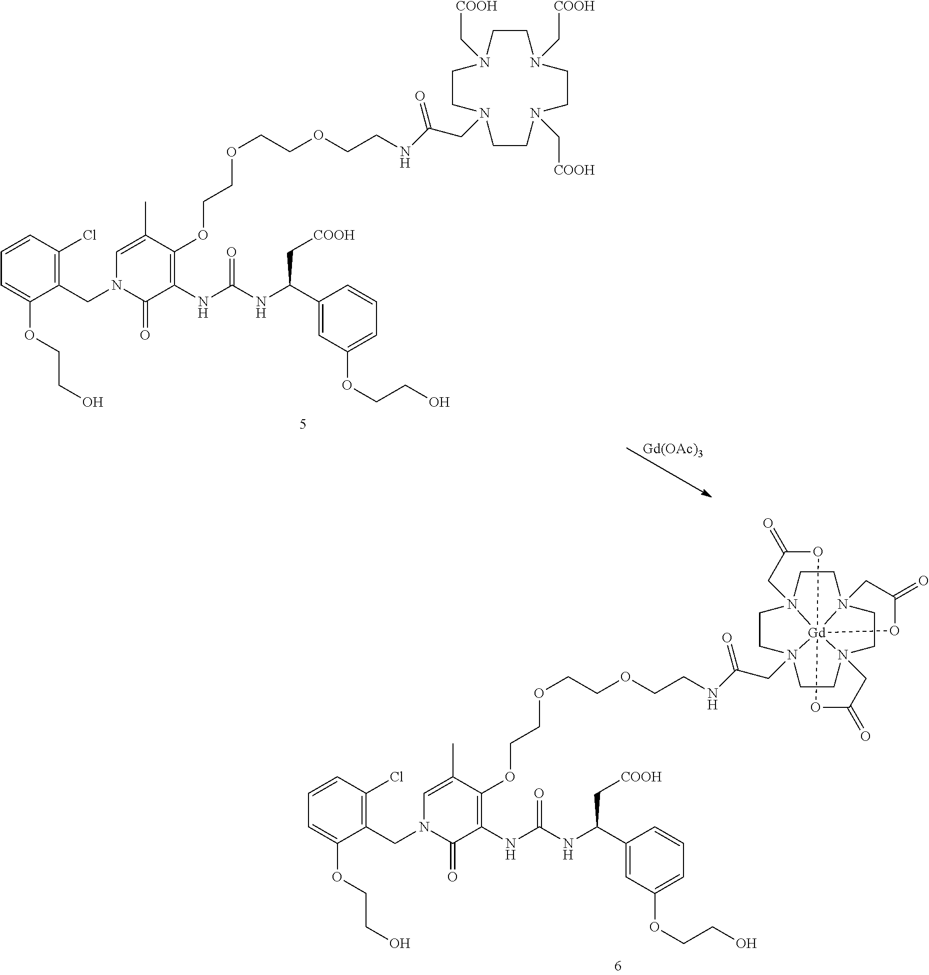

Step One: To a solution of DOTA tris(tert-butyl ester) (compound 1, 504 mg, 0.88 mmol) in dichloromethane (59 mL) at room temperature under argon, N-hydroxysuccinimide (162 mg, 1.41 mmol) and N-(3-dimethylaminopropyl)-N′-ethylcarbodiimide hydrochloride (EDCI, 270 mg, 1.41 mmol) were added sequentially. The resulting mixture was stirred at room temperature overnight, then was extracted with 1:1 brine:water (twice), 1:1 saturated aqueous sodium bicarbonate:water, and brine. The organic layer was dried over magnesium sulfate, filtered and concentrated under reduced pressure to give compound 2 (588 mg) as a light yellow-orange solid.

Step Two: To a flask containing compound 2 (161 mg, 0.24 mmol) at room temperature under argon, a solution of compound 3 (209 mg, 0.24 mmol) and triethylamine (0.045 mL, 0.32 mmol) in N,N-dimethylformamide (DMF, 14 mL) was added by cannula along with a DMF (2 mL) rinse. The mixture was stirred at room temperature overnight, then was concentrated. The residue was purified by preparative reverse phase HPLC (Symmetry Shield RPC18, 30×250 mm column, 7 μm, 20-80% acetonitrile in 0.1% trifluoroacetic acid in water, 40 mL/minute, loaded in ˜1:1 methanol:water with 5 drops acetic acid). Fractions containing pure compound 3 were combined and the acetonitrile was partially removed by rotary evaporation. The resulting solution was frozen and lyophilized to give compound 4 (125 mg) as a fluffy off-white solid. Structure confirmed by LCMS.

Step Three: To a solution of compound 4 (120 mg, 0.083 mmol) in anhydrous dichloromethane (6 mL) at room temperature under argon, trifluoroacetic acid (6 mL) was added.

The resulting mixture was stirred at room temperature for 6 hours, then was concentrated under reduced pressure. The residue was re-dissolved in dichloromethane and concentrated (3 times). The residue was purified by preparative reverse phase HPLC (Symmetry Shield RPC18, 30×250 mm column, 7 μm, 0-40% acetonitrile in 0.1% trifluoroacetic acid in water, 40 mL/minute). Fractions containing pure compound 4 were combined and the acetonitrile was removed by rotary evaporation. The resulting solution was frozen and lyophilized to give compound 5 (75 mg) as a fluffy off-white solid. Structure confirmed by LCMS.

Step Four: To a solution of compound 5 (72 mg, 0.066 mmol) in water (2 mL) at room temperature, Gd(OAc)₃.xH₂O (26.8 mg, 0.066 mmol, x=4 based on certificate of analysis) was added. The homogeneous solution was stirred at room temperature overnight. LCMS indicated partial complexation. To the resulting solution, pyridine (0.020 mL, 0.264 mmol) was added by syringe, and the mixture was stirred for 1.5 hours. LCMS indicated complete uptake of gadolinium, and the mixture was directly purified by reverse phase chromatography (Biotage, SNAP 30 C18 cartridge, 0-50% acetonitrile in water). The center cut of the eluting peak was concentrated under reduced pressure to remove the acetonitrile, then was lyophilized to give compound 6 (36 mg) as a fluffy white solid. LCMS showed complete complexation.

In an alternate synthesis of compound 4, acid activation has been performed by combining compound 1 with 1.1 equivalents of ethyl chloroformate in dichloromethane at room temperature in the presence of a tertiary amine base. The resulting solution is diluted with ether and decanted into a solution of compound 3 in dichloromethane with additional amine base added.

Other methods of acid activation may include CDI, HBTU, TSTU, among others.

### Example 5

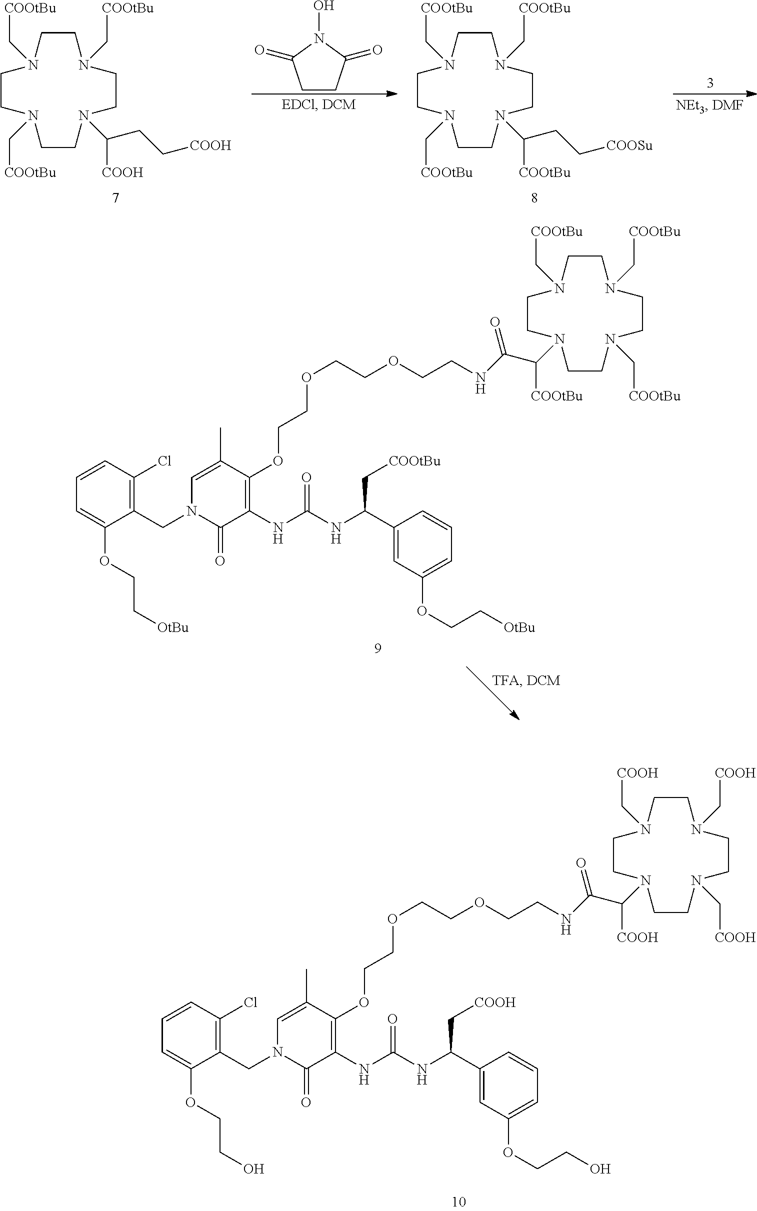

Step One: Step one from example one was followed using compound 7 (103.4 mg, 0.148 mmol) to give compound 8 (109 mg) as an off-white foam.

Step Two: Step two from example one was followed using compound 8 (109 mg, 0.136 mmol) to give compound 9 (190 mg) as a fluffy white powder. Structure confirmed by LCMS.

Step Three: Step three from example one was followed using compound 9 (190 mg, 0.121 mmol) to give compound 10 (120 mg) as a fluffy white powder. Structure confirmed by LCMS.

Alternatively, compound 9 was synthesized by treating compound 8 with 1.1 equivalents of ethyl chloroformate in dichloromethane at room temperature in the presence of a tertiary amine base. The resulting solution is diluted with ether and decanted into a solution of compound 3 in dichloromethane with additional amine base added.

Other methods of acid activation may include CDI, HBTU, TSTU, among others.

### Example 6

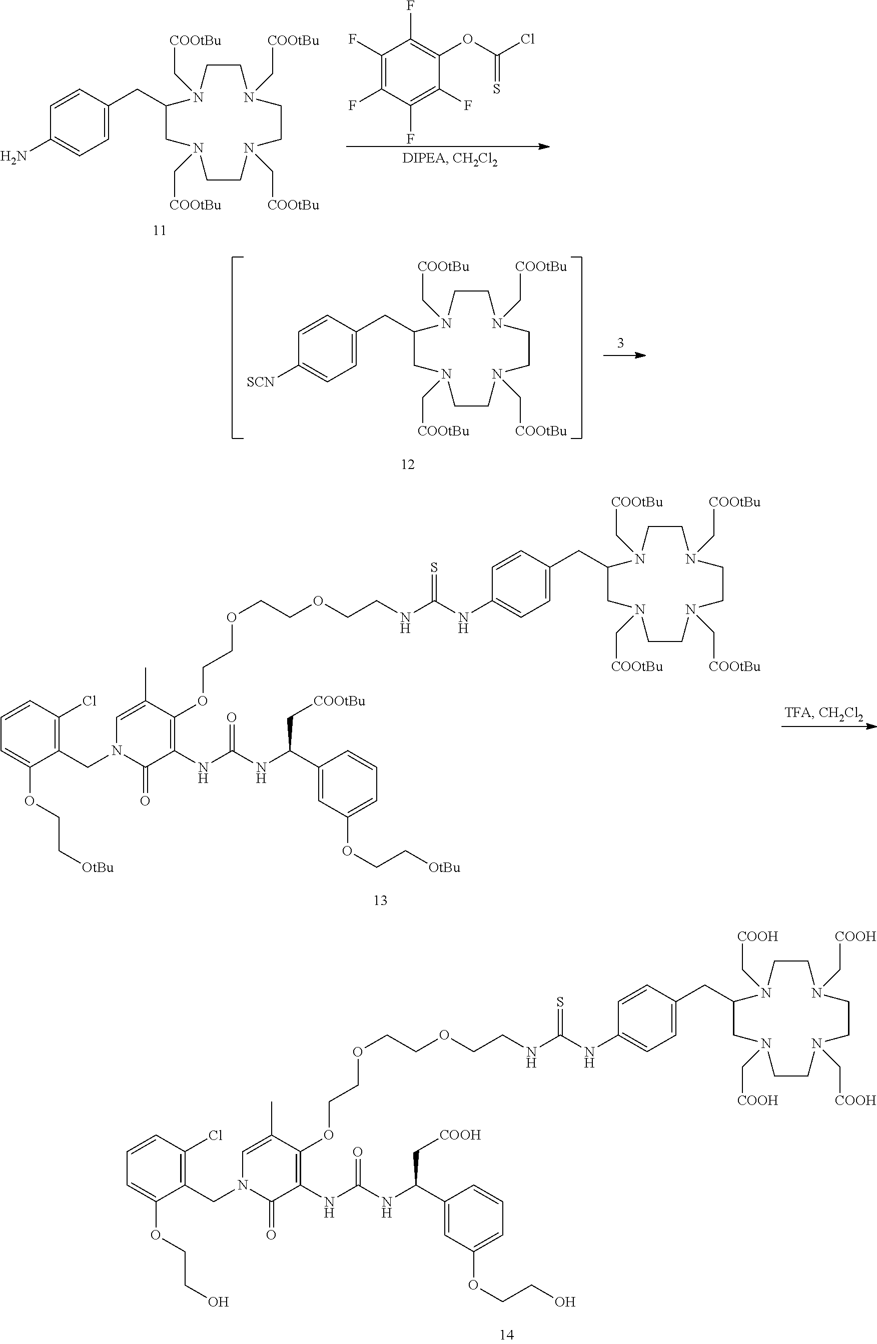

Step One: The TFA salt of compound 11 was converted to compound 11 by dissolving in dichloromethane, washing with aqueous sodium hydroxide (1 N) and 1:1 brine:water followed by drying the organic layer over MgSO₄, filtering and concentrating. The resulting freebase compound 11 (33 mg, 0.045 mmol) was dissolved in dichloromethane (1 mL) at room temperature under argon, and DIPEA (12 μL, 0.067 mmol) and pentafluorophenyl chlorothioformate (13 μL, 0.067 mmol) were added sequentially. After two hours, a small aliquot was withdrawn, concentrated and analyzed by LCMS, which showed complete consumption of compound 11 along with the in situ formation of compound 12. To the reaction mixture, compound 3 (80 mg, 0.091 mmol) was added. The mixture was stirred at room temperature overnight, then was heated to 35° C. for 3 hours, then was concentrated. The residue was purified by preparative reverse phase HPLC (Symmetry Shield RPC18, 30×250 mm column, 7 μm, 20-80% acetonitrile in 0.1% trifluoroacetic acid in water, 40 mL/minute, loaded in ˜3:1 methanol:water with 5 drops acetic acid). The fraction containing the desired compound was frozen, then lyophilized to give compound 13 (17 mg) as a fluffy white solid.

Step Two: Step three from example one was followed using compound 13 (17 mg, 0.010 mmol) to give compound 14 as a fluffy white powder. Structure confirmed by LCMS.

### Example 7

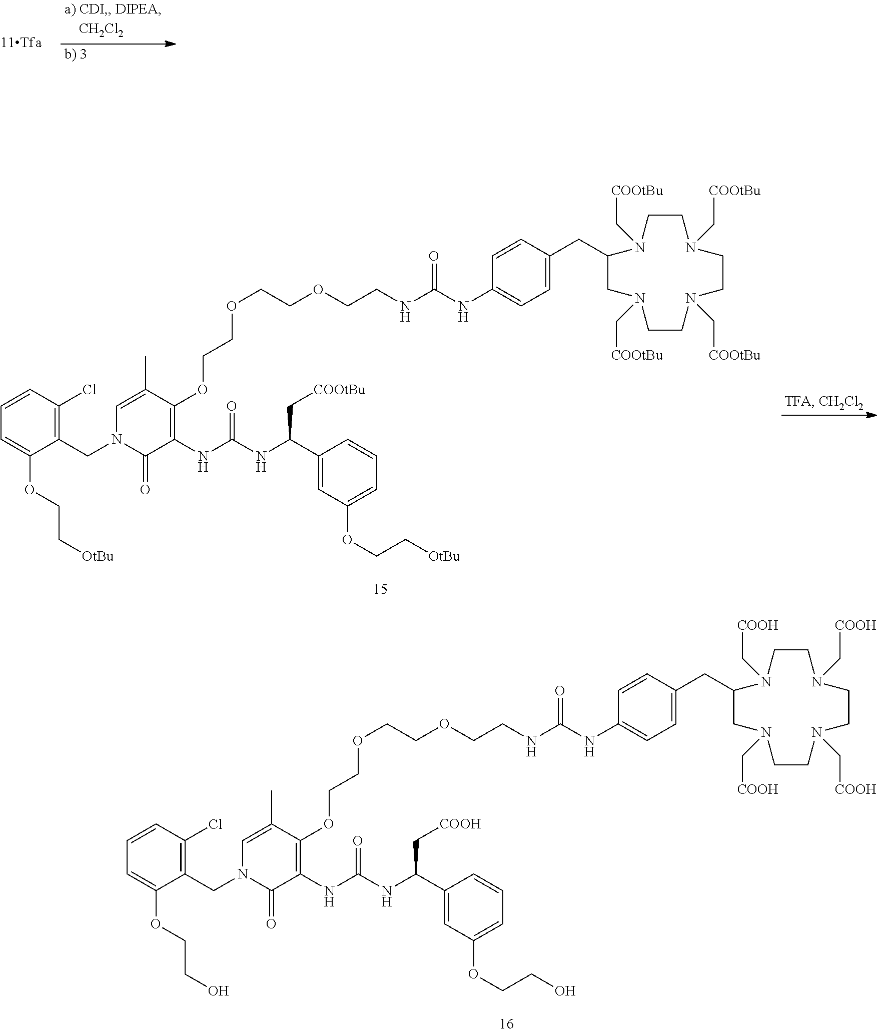

Step One: To a solution of compound 11.Tfa (28.8 mg, 0.034 mmol) in tetrahydrofuran (0.2 mL) and DIPEA (0.1 mL), 1,1′-carbonyldiimidazole (CDI, 5.2 mg, 0.032 mmol) was added. The resulting mixture was stirred at room temperature for 30 minutes, then compound 3 (25.2 mg, 0.029 mmol) was added. The resulting mixture was stirred at room temperature overnight, then was concentrated. The residue was purified on a Biotage Isolera 4 (SNAP KPNH cartridge, 0 to 20% methanol in ethyl acetate) to give compound 15 (7.7 mg).

Step Two: Step three from example one was followed using compound 15 (7.7 mg, 0.0047 mmol) to give compound 16 (2.8 mg) as a fluffy white solid. Structure confirmed by LCMS.

### Example 8

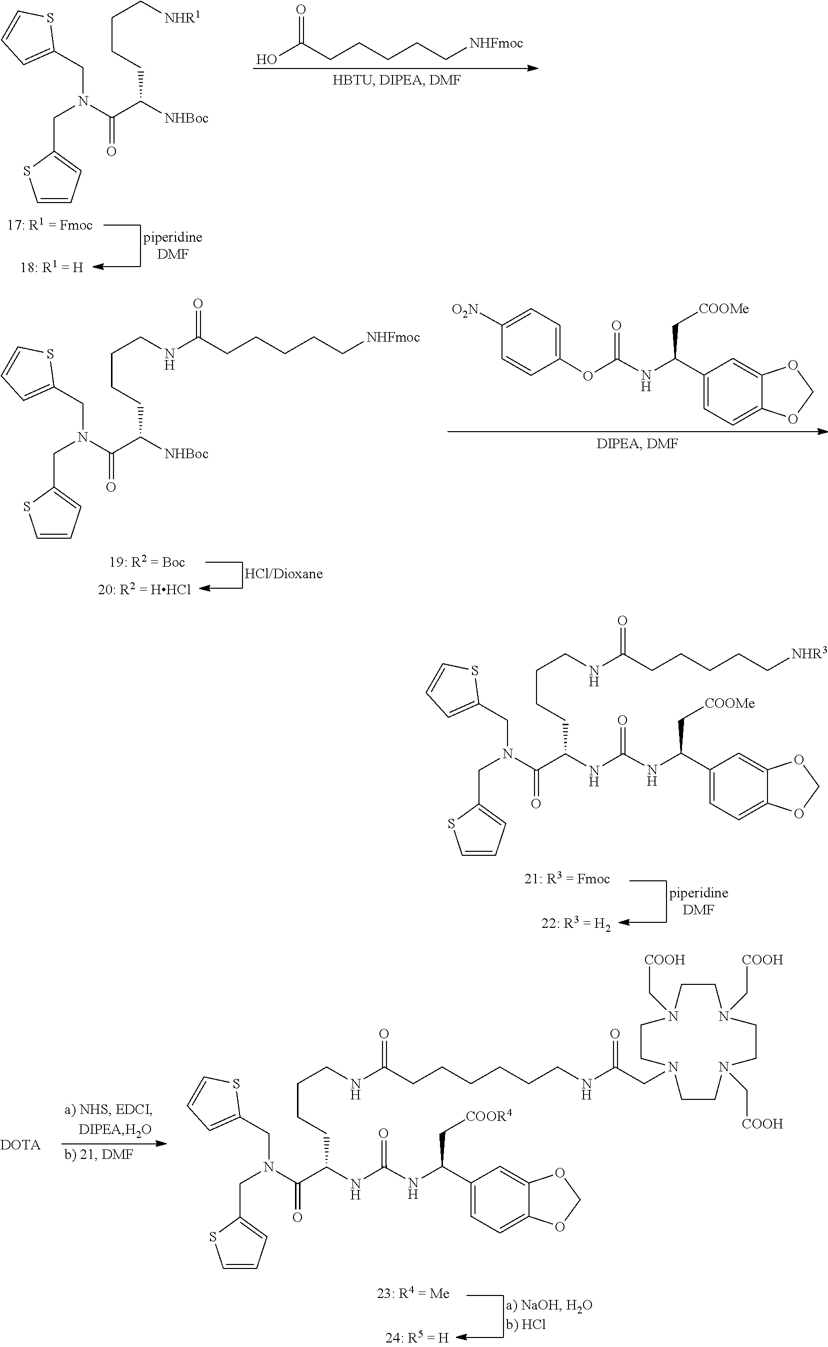

Step One: A solution of compound 17 (871 mg, 1.32 mmol), prepared according to procedures described in U.S. Pat. No. 6,194,448, incorporated herein by reference, in DMF (5.2 mL) and piperidine (0.52 mL) was stirred at room temperature for 1 hour. The resulting mixture was taken up in acetonitrile and was extracted twice with hexanes. The acetonitrile layer was concentrated to about 5 mL, then was partitioned between ethyl acetate and water. The aqueous layer was extracted with ethyl acetate, then the combined organic layers were washed with water (four times) and brine. The organic layer was dried over MgSO₄, filtered and concentrated to give compound 18 (589 mg) as an oil. This contained a small amount of impurities related to the Fmoc group but was used without purification.

Step Two: To a solution of crude compound 18 (566 mg, 1.29 mmol theoretical) and 6-((((9H-fluoren-9-yl)methoxy)carbonyl)amino)hexanoic acid (544 mg, 1.54 mmol) in DMF (4 mL) at room temperature under argon, DIPEA (0.29 mL, 1.68 mmol) and HBTU (584 mg, 1.54 mmol) were added sequentially. The mixture was stirred at room temperature for 3 hours, then was diluted with 1:1 hexanes:ethyl acetate, and washed with aqueous HCl (2N), water (4 times), saturated aqueous NaHCO₃, and brine. The organic layer was dried over MgSO₄, filtered and concentrated. The residue was filtered through a short pad of silica gel, eluting with 1:1 hexanes:ethyl acetate followed by 1:1 hexanes:ethyl acetate plus 5% methanol to give compound 19 (850 mg) as an off-white solid.

Step Three: To a flask containing compound 19 (850 mg, 1.10 mmol) under argon, a solution of HCl in dioxane (4.0 M, 3 mL, 12 mmol) was added. The resulting solution was stirred at room temperature overnight, then the excess HCl was removed by bubbling argon through the reaction mixture. The reaction mixture was concentrated, and the residue was taken up in dichloromethane and concentrated (twice) to give compound 20 (810 mg) as a orange-brown solid.

Step Four: To a solution of compound 20 (233 mg, 0.31 mmol) in dichloromethane (1 mL) at room temperature under argon, DIPEA (0.113 mL, 0.66 mmol) and (S)-methyl 3-(benzo[d][1,3]dioxol-5-yl)-3-(((4-nitrophenoxy)carbonyl)amino)propanoate (136 mg, 0.35 mmol), prepared according to procedures described in U.S. Pat. No. 6,194,448, incorporated herein by reference, were added. The resulting mixture was stirred at room temperature overnight, then was diluted with ethyl acetate and washed with 1:1 saturated aqueous NaHCO₃:water, water (eight times) and brine. The organic layer was dried over MgSO₄, filtered and concentrated. The residue was purified by silica gel chromatography, eluting with 1:1 hexanes:ethyl acetate increasing to ethyl acetate and finally 19:1 ethyl acetate:methanol to give compound 21 (174 mg) as an off-white solid.

Step Five: A solution of compound 21 (172 mg, 0.187 mmol) in DMF (1.5 mL) and piperidine (1.5 mL) was stirred at room temperature overnight. The mixture was diluted with ethyl acetate and extracted with 1:1 saturated aqueous NaHCO₃:water, water (four times) and brine. The organic layer was dried over MgSO₄, filtered and concentrated. The residue was taken up in 2:3 acetonitrile:0.1% TFA in water and filtered through a cotton plug to remove the insoluble material. The filtrate was further filtered through a Sep-Pak cartridge, rinsing with 2:3 acetonitrile:0.1% TFA in water. The filtrate was concentrated to remove the acetonitrile, and the mixture was then re-filtered though another Sep-Pak cartridge, eluting with 1:4 acetonitrile:0.1% TFA in water. The filtrate was diluted with saturated aqueous NaHCO₃, and extracted twice with dichloromethane. The organic layers were dried over MgSO₄, filtered and concentrated. The residue was further purified by reverse phase HPLC (Symmetry Shield RPC18, 19×150 mm column, 7 μm, 30-80% methanol in 0.1% trifluoroacetic acid in water, 15 mL/minute). Fractions containing the desired material were made basic with saturated aqueous NaHCO₃, and extracted twice with dichloromethane. The organic layers were dried over MgSO₄, filtered and concentrated to give compound 22 (38 mg) as a bright yellow solid.

Step Six: To a solution of DOTA (82 mg, 0.204 mmol) in deionized water (0.5 mL), N-hydroxysuccinimide (NHS, 29 mg, 0.25 mmol), DIPEA (71 μL, 0.41 mmol) and EDCI (49 mg, 0.25 mmol) were added sequentially. The resulting mixture was stirred for 30 minutes, then a solution of compound 22 (36 mg, 0.051 mmol) in DMF (0.34 mL) was added by cannula along with a 0.1 mL DMF rinse. The mixture was stirred overnight, then was diluted with methanol (1 mL) and water (1 mL) and the solution was acidified by dropwise addition of aqueous HCl (2N). This mixture was directly purified by reverse phase HPLC (Symmetry Shield RPC18, 30×250 mm column, 7 μm, 20-70% methanol in 0.1% trifluoroacetic acid in water, 40 mL/minute). A center cut of the peak for the desired compound was concentrated to remove the methanol, then was frozen and lyophilized to give compound 23 as a fluffy white powder.

Step Seven: A solution of compound 23 was dissolved in aqueous sodium hydroxide (2N, 1 mL) and was stirred for 4 hours. The mixture was acidified with aqueous HCl (2N), then was filtered through a Sep-Pak. The fraction containing the desired compound was lyophilized to give compound 24 (4.2 mg) as a fluffy white powder.

### Example 9

A chelator with three acidic coordination sites (tridentate) can be attached via formation of urea.

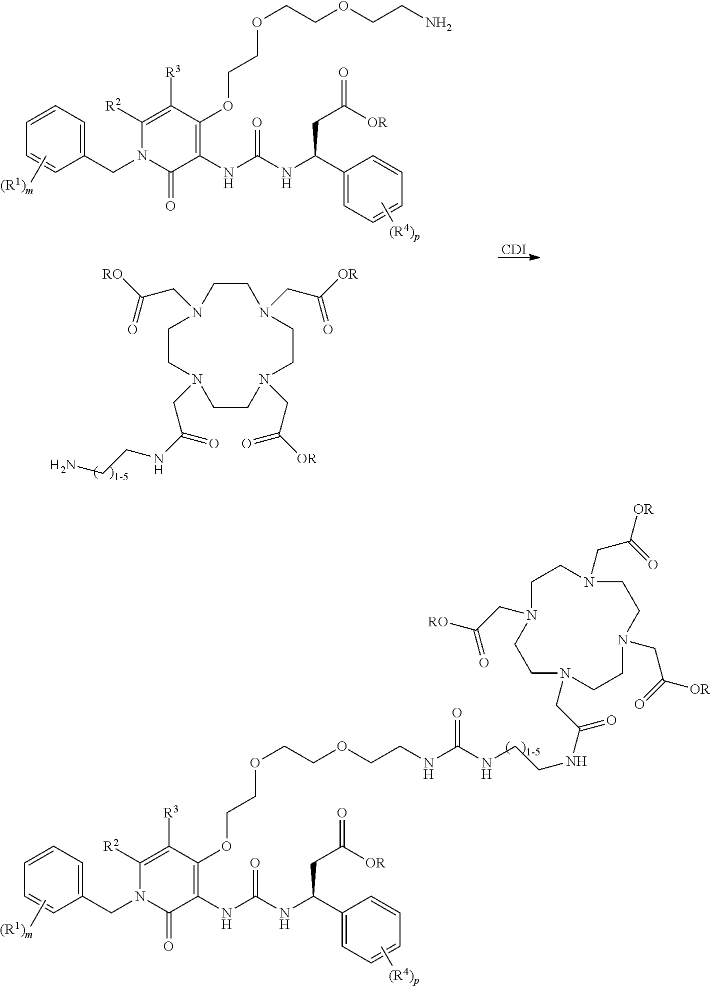

### Example 10

A conjugate with a chelator with three acidic coordination sites (tridentate) can be made via Click chemistry.

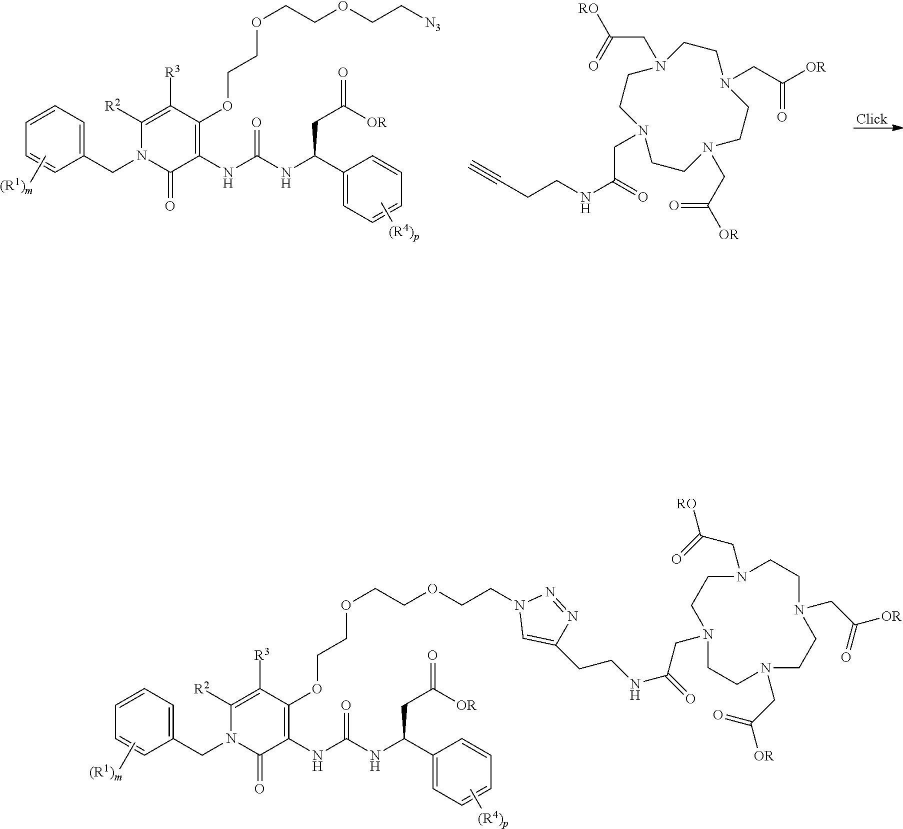

### Example 11

A conjugate with a chelator with four acidic coordination sites (tetradentate) can be made via Click chemistry.

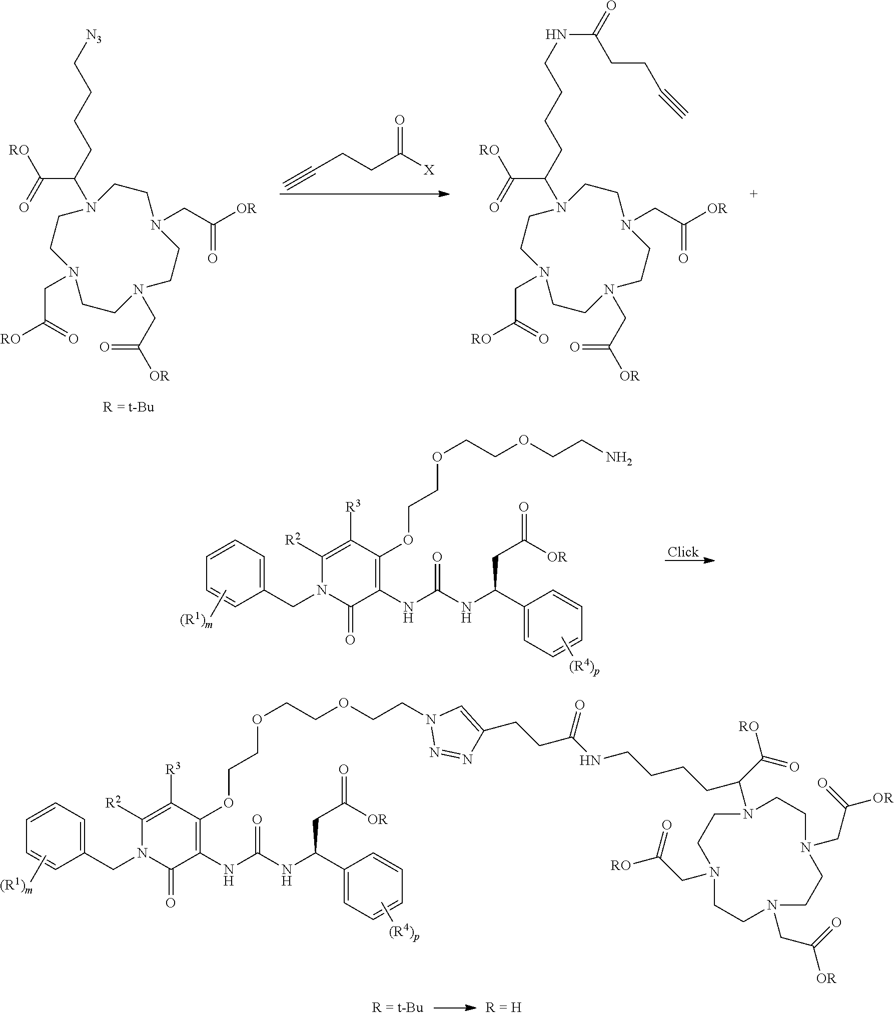

### Example 12

A chelator with three acidic coordination sites (tridentate) can be attached via formation of an amide.

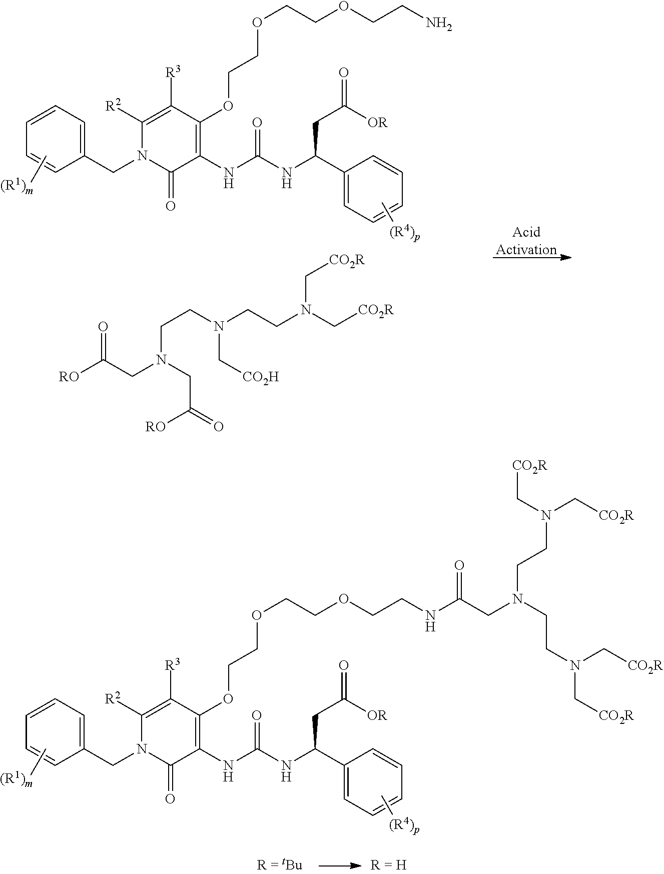

### Example 13

A conjugate with a chelator with five acidic coordination sites (pentadentate) can be made via the formation of urea.

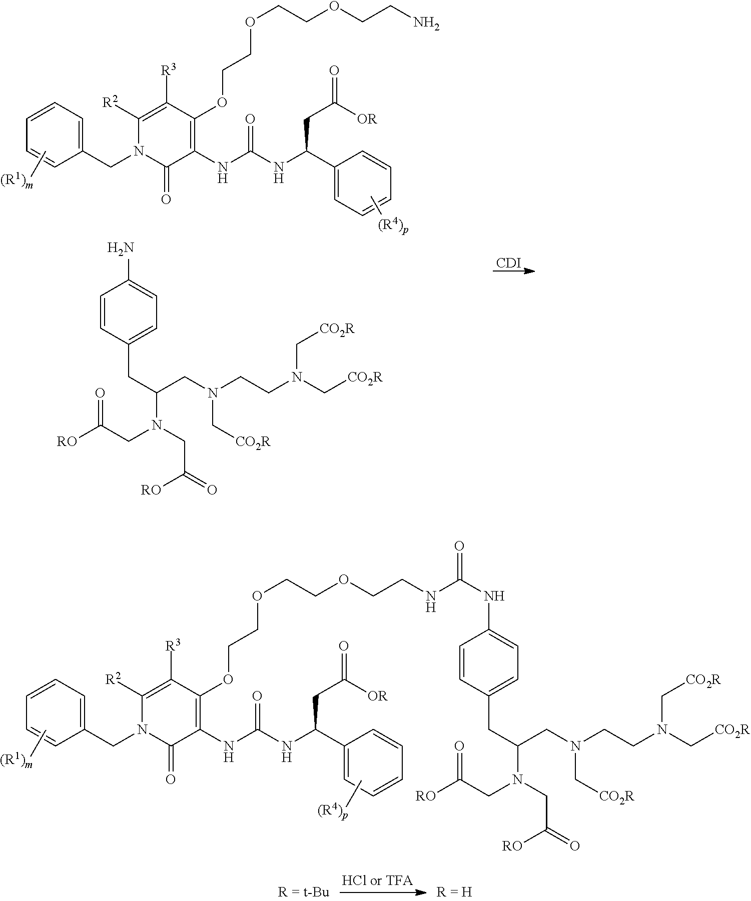

### Example 14

A conjugate with a chelator with five acidic coordination sites (pentadentate) can be made via the formation of thiourea.

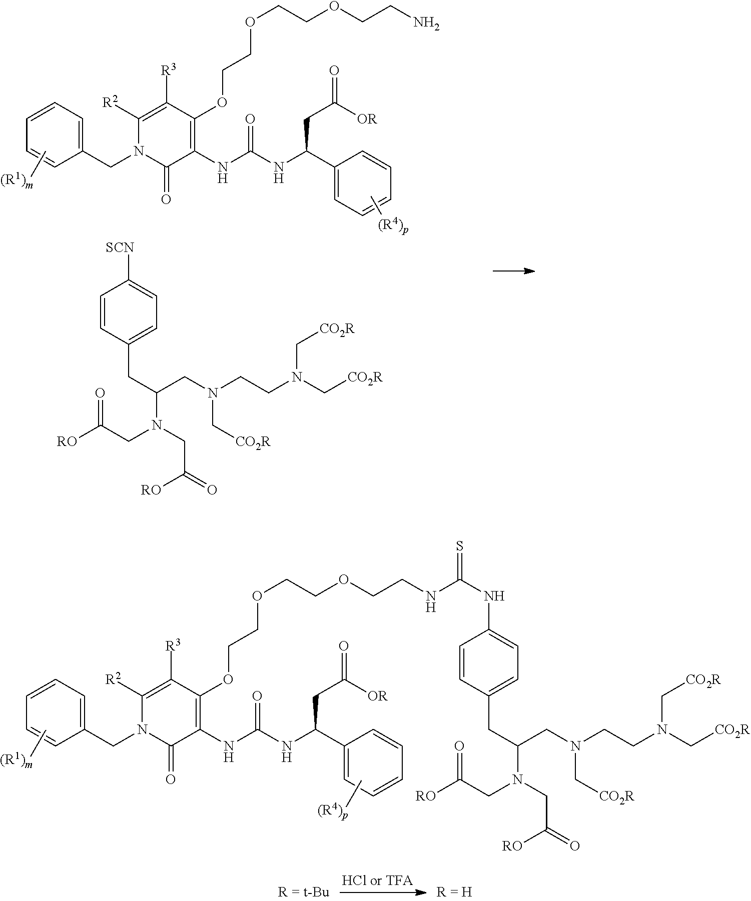

### VLA-4 Antagonist-Linker-Dye Conjugates

In the examples of FIGS. 15 to 20, VLA-4 Antagonist-linker-dye conjugates were also prepared from commercially available dyes with activated carboxylic acids, typically the Su ester, for example, by forming amides using similar methods from intermediates described herein. Stereoisomers may alternatively be synthetized.

### Example 15 (THI516)

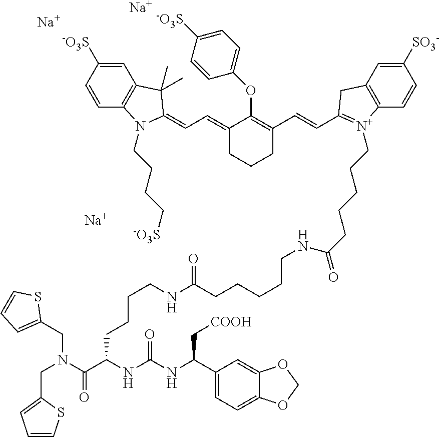

| Activity (IC₅₀) | | | | |
|---|---|---|---|---|
| Cell Type | Substrate | Cations | Species | General Notes |
| 6.5 ± 3.5 nM | C5-1 | Mn | Human | Intravital microscopy |
| (n = 4) | | | | showed high specific |
| K562(α4β1) | | | | binding to what appears |
| 186.1 nM | C5-1 | Ca/Mg | Human | to be extracellular |
| (n = 1) | | | | matrix in the lung. |
| K562(α4β1) | | | | There was no staining |
| 6 ± 1 nM | Flow | Mn | Human | of murine lymphocytes |
| (n = 3) | Cytometry | | | in the lung. |
| K562(α4β1) | | | | |

### Example 16 (THI526)

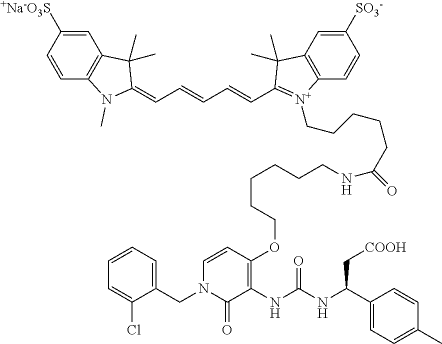

### Example 17 (THI528)

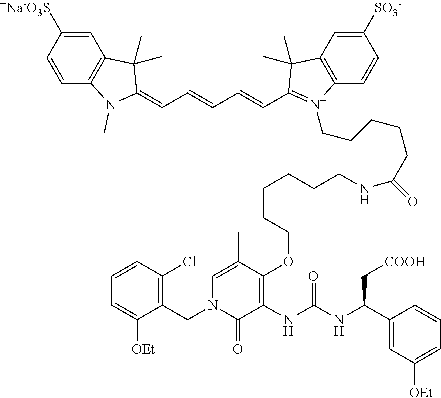

a4b1 (IC₅₀=519±97 pM; n=3).

### Example 18 (THI509)

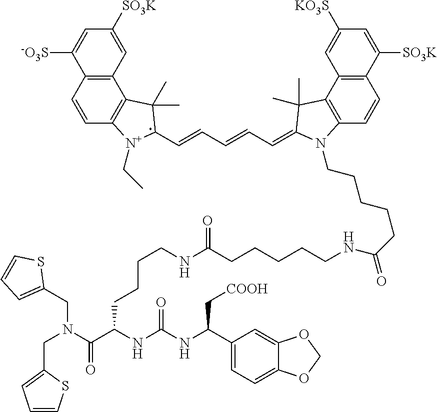

### Example 19 (THI540)

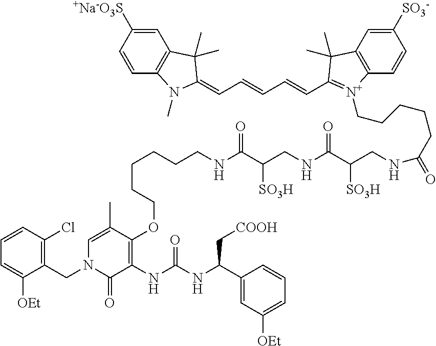

### Example 20 (THI552)

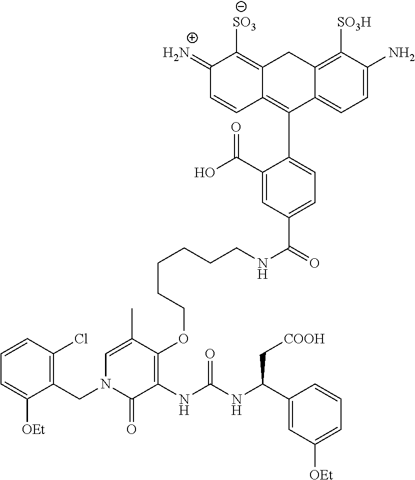

with Alexa Fluor 488

### Synthesis of LFA-1 Antagonist Intermediates

LFA-1 antagonist intermediates can be synthetized as shown in examples 21-25. Stereoisomers may alternatively be synthetized.

### Example 21

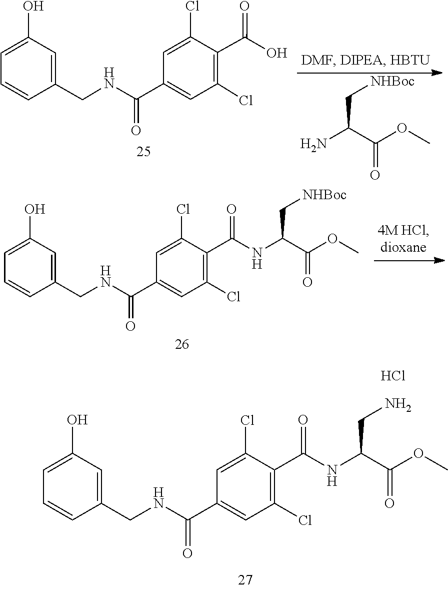 [SMILES: O=C(NCc1cccc(O)c1)c1cc(Cl)c(C(=O)O)c(Cl)c1 ; COC(=O)[C@@H](N)CNC(=O)OC(C)(C)C ; COC(=O)[C@H](CNC(=O)OC(C)(C)C)NC(=O)c1c(Cl)cc(C(=O)NCc2cccc(O)c2)cc1Cl ; Cl ; COC(=O)[C@H](CN)NC(=O)c1c(Cl)cc(C(=O)NCc2cccc(O)c2)cc1Cl]

Step 1: To a solution of the carboxylic acid (compound 25, 1.62 g, 6.8 mmol), prepared according to procedures described in U.S. Pat. No. 7,217,728, incorporated herein by reference, in DMF (15 mL) was added sequentially diisopropylethylamine (0.9 mL), HBTU (2.7 g) and the commercially available BOC-protected-amino ester (1.64 g) under argon. The resulting mixture was heated at 80° C. Upon completion of the reaction, the mixture was partitioned between 1:1 hexanes:ethyl acetate (2×) and dilute HCl (<0.5M). The combined organic layer was washed with brine and dried over sodium sulfate, then filtered through a pad of course silica gel washing with 1:1 hexanes:ethyl acetate. The filtrate was concentrated to give compound 26.

Step 2: Crude compound 26 from step 1 was dissolved in HCl in 1,4-dioxane (4 M, 8 mL) at room temperature overnight. The excess HCl was blown off under a stream of air, and the residue was purified on C18 reverse phase chromatography using a gradient elution to give compound 27.

### Example 22

The following example is representative of linkers that may be installed on the ring as shown. One skilled in the art would recognize the generality of the method used to install linkers similar to the one shown. In order to ensure that the DOTA moiety does not interfere in the bound state, the embodiments are at least 2 atoms in length, preferably 4 atoms or more.

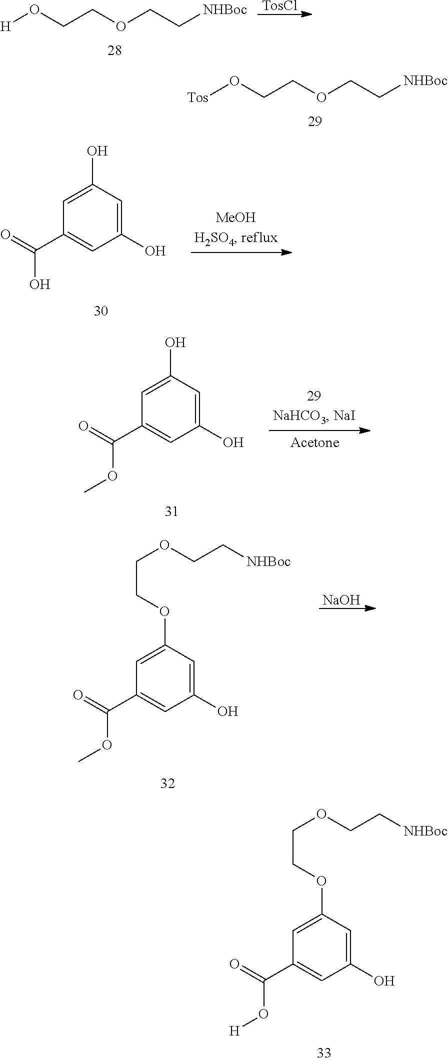

Step 1: Commercially available t-butyloxycarbonyl protected amino-alcohol 28 (4.88 g) is dissolved in dichloromethane (10 0 mLs and treated with triethylamine (3.6 mL, 26.1 mmol), catalytic 4-(N,N-dimethylamino)pyridine (10 mol %) and p-toluenesulfonyl chloride (4.29 g, 0.95 equivalents). The mixture was stirred overnight, then concentrated to dryness, re-suspended in diethyl ether and filtered. The solvent layer was loaded directly onto Silica gel and eluted with 2:1 hexanes:ethyl acetate to give compound 29.

Step 2: Compound 31 was prepared from compound 30 by Fisher esterification in methanol.

Step 3. The methyl ester compound 31 (3.8 g) was alkylated by dissolving in acetone (50 mL) and treating with potassium carbonate (1.1 g) and catalytic sodium iodide. To this solution was added compound 29 (3.8 g) and the resulting mixture was brought to reflux. Upon completion of the reaction the solvent was decanted and evaporated under reduced pressure. The residue was purified on silica gel to give compound 32 as well as some of the di-substituted product.

Step 4: To a solution of compound 32 (0.32 g) in acetonitrile (5 mL) was added an aqueous solution of sodium hydroxide (2N, 1.5 mL). Upon completion the reaction, the mixture was diluted with water and extracted with diethyl ether. The ether layer was set aside and the aqueous layer was acidified with 2N HCl and extracted with ethyl acetate. The aqueous was washed twice more with ethyl acetate. The ethyl acetate layers were combined, washed with brine, dried over magnesium sulfate filtered and concentrated to give compound 33. The resulting material was used without purification.

### Example 23

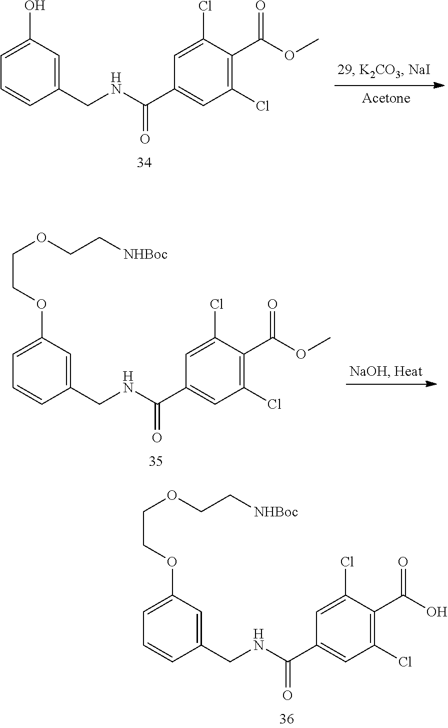 [SMILES: COC(=O)c1c(Cl)cc(C(=O)NCc2cccc(O)c2)cc1Cl ; COC(=O)c1c(Cl)cc(C(=O)NCc2cccc(OCCOCCNC(=O)OC(C)(C)C)c2)cc1Cl ; CC(C)(C)OC(=O)NCCOCCOc1cccc(CNC(=O)c2cc(Cl)c(C(=O)O)c(Cl)c2)c1]

Step 1: To a solution of compound 34 (2.3 g, 4.93 mmol), prepared according to procedures described in U.S. Pat. No. 7,217,728, which is incorporated by reference herein, in acetone (20 mL) was added compound 29 (1.94 g), potassium carbonate (1.02 g 7.4 mmol) and sodium iodide (catalytic). The resulting mixture was refluxed overnight, cooled, filtered and concentrated to dryness. The residue was purified on silica gel eluting with 3:1 hexanes:ethyl acetate to give compound 35.

Step 2: To a solution of compound 35 (0.91 g, 1.68 mmol) in acetonitrile (7 mL) was added aqueous sodium hydroxide (2N). A small amount of methanol was added to make a homogeneous solution. Upon completion of the reaction, the mixture was diluted with water and extracted with diethyl ether. The ether layer was set aside and the aqueous layer was acidified with 2N HCl and extracted with ethyl acetate. The aqueous was washed twice more with ethyl acetate.

The ethyl acetate layers were combined, washed with brine, dried over magnesium sulfate filtered and concentrated to give compound 36.

### Example 24

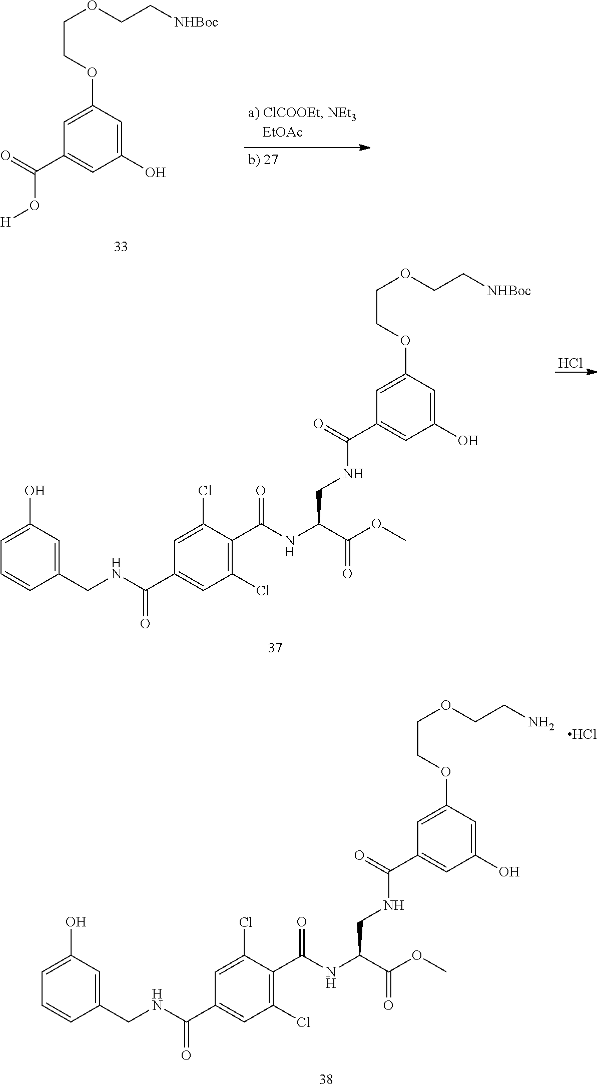 [SMILES: CC(C)(C)OC(=O)NCCOCCOc1cc(O)cc(C(=O)O)c1 ; COC(=O)[C@H](CNC(=O)c1cc(O)cc(OCCOCCNC(=O)OC(C)(C)C)c1)NC(=O)c1c(Cl)cc(C(=O)NCc2cccc(O)c2)cc1Cl ; Cl ; COC(=O)[C@H](CNC(=O)c1cc(O)cc(OCCOCCN)c1)NC(=O)c1c(Cl)cc(C(=O)NCc2cccc(O)c2)cc1Cl ; Cl]

Step 1: To a solution of compound 33 (89.7 mg, 0.287 mmol) in ethyl acetate (1.5 mL), triethylamine (0.13 mL, 1.7 mmol) and ethyl chloroformate (0.30 mL) were added. The resulting mixture was allowed to stand overnight before being filtered through diatomaceous earth and washed with ethyl acetate. The filtrate was treated with compound 27 (1.0 equivalent) with additional triethylamine (0.13 mL). Upon completion of the reaction was purified by silica gel chromatography eluted with hexanes:ethyl acetate mixtures to give compound 37.

Step 2: To a flask containing compound 37 (128 mg) in an ice bath, 4M HCl in 1,4-dioxane was added. The resulting mixture was allowed to warm to room temperature overnight and the excess HCl was blown off with a stream of air and the mixture was concentrated under reduced pressure to give compound 38 (113 mg).

### Example 25

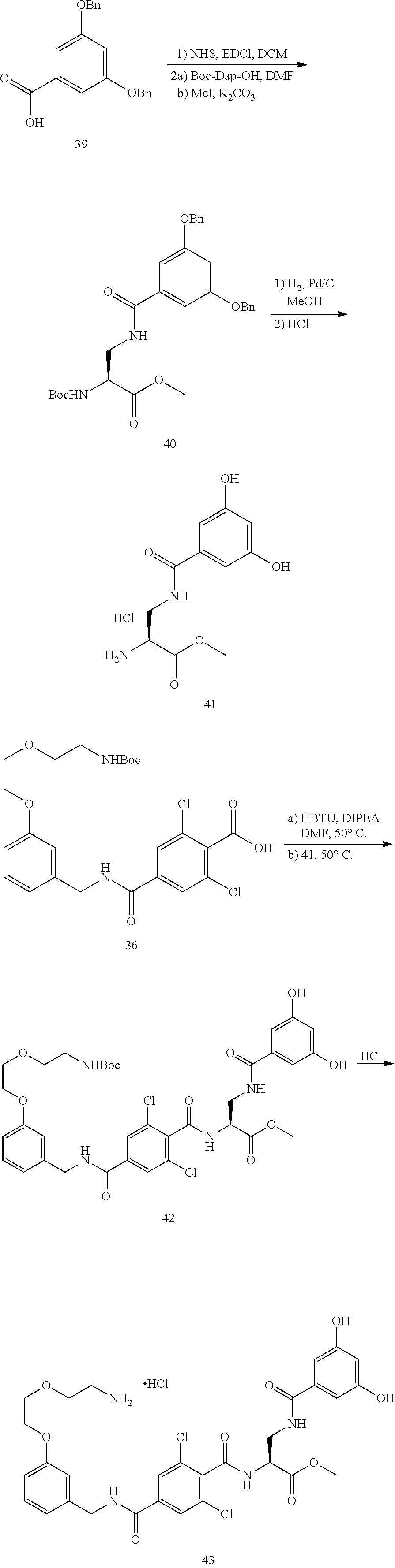

Step 1: To a solution of compound 39 (prepared by Fisher esterification of 3,5-dihydroxybenzoic acid, benzylation of the two phenols, followed by ester hydrolysis, 655 mg, 4.88 mmol) in dichloromethane, EDCI (1.40 g, 7.37 mmol) and N-hydroxysuccinimide (NHS) (0.620 g) were added. Upon completion of the reaction (TLC), the solution was concentrated, then taken up in ethyl acetate washed with water and brine, dried over sodium sulfate, filtered and concentrated. The residue was purified on silica gel using hexanes:ethyl acetate 3:1 to 2:1 gradient to give the O-Su ester. To a solution of the Su ester (325 mg) in dichloromethane (4 mL) and DMF (0.5 mL), Boc-DAP-OH (169 mg, 1.1 equivalents, 0.829 mmol) was added. The mixture was stirred overnight. Thereafter, the DMF was increased to 2 mLs and potassium carbonate was added (362 mgs) followed by excess methyl iodide (1.5 equivalents). Stirring was continued for 24 hours and the resulting mixture was partitioned between ethyl acetate and water. The organic layer was washed with brine, dried over sodium sulfate, filtered and concentrated to give compound 40.

Step 2: The resulting crude compound 40 (455 mg) was dissolved in methanol (8 mL) and the atmosphere was exchanged for argon via vacuum to argon flow. Palladium on carbon (10% on Carbon dry weight basis, 50% water, 0.94 g) was added and the atmosphere was exchanged for hydrogen via vacuum to hydrogen flow. The mixture was heated at 50° C. for 18 hours. The suspension was filtered through diatomaceous earth and concentrated to dryness. The residue was brought up in 4M HCl in 1,4-dioxane (4 mL) and was stirred overnight. A stream of air was used to blow off the excess HCl and the mixture was concentrated to dryness under reduced pressure to give compound 41.

Step 3: To a solution of compound 36 (0.8311 g 1.576 mmol) in DMF (3 mL), diisopropylethylamine (1.1 mL, 6.3 mmol) and HBTU (657 mg, 1.73 mmol) were added. The mixture was introduced into an oil bath regulated to 50° C. A solution of compound 41 (247.8 mgs, 0.975 mmol) in 1 mL of DMF (1 mL) was then added via syringe. After stirring overnight, the mixture was partitioned between ethyl acetate and brine containing dilute HCl. The organic layer was dried over sodium sulfate, filtered and concentrated to give compound 42.

Step 4: To a flask containing compound 42 (0.1035 g) was added 4M HCl in 1,4-dioxane. The mixture was stirred at room temperature overnight. The excess HCl was then blown off with a stream of air and the solvent was removed by rotary evaporation. The residue was purified by C18 reverse phase chromatography using acetonitrile:water mixtures as the eluent to give compound 43.

### LFA-1 Antagonist-Linker-Chelator Conjugates

The chelator moieties previously discussed can be useful in making conjugates based on LFA-1 antagonist in accordance with the following structures and/or stereoisomers thereof:

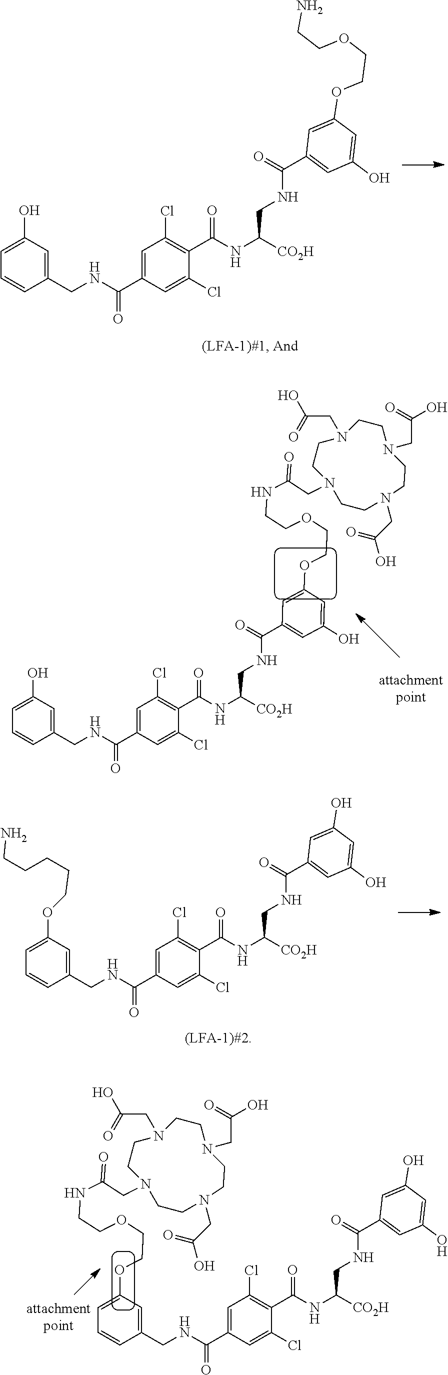

Where (LFA-1)#1 or #2 refer to the phenolic attachment point to be used on the LFA-1 antagonist.

The linker chains outlined in the VLA-4 antagonist-linker-chelator conjugates are likewise available in these series of compounds, e.g. azido functionality in place of an amine for Click chemistry. The LFA-1 antagonist intermediates are combined with appropriate metal chelating ligands as shown below. The coupling to these chelators is well known in the literature and may be produced accordingly.

The following examples 26 to 35 show multi-dentate arrangements of LFA-1 antagonist conjugates. Stereoisomers may alternatively be synthetized.

### Example 26

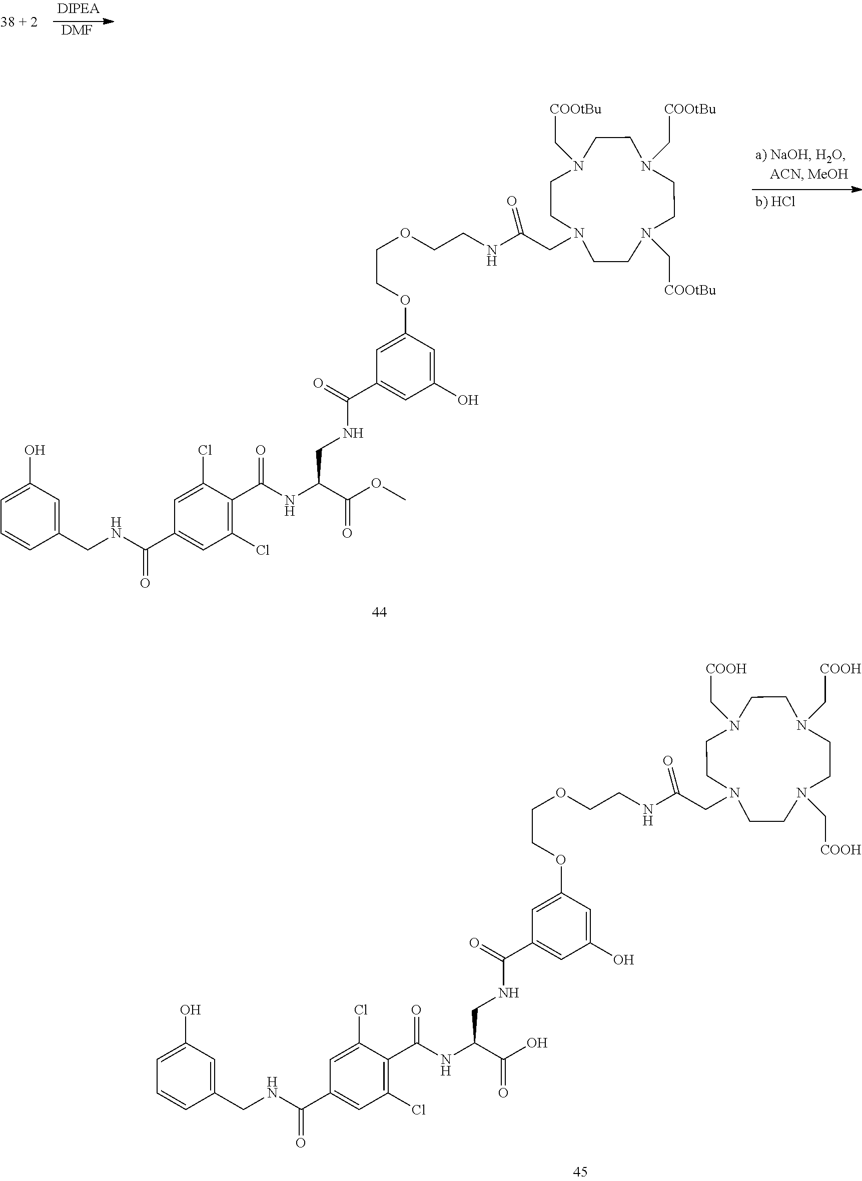

Step 1: To a solution of compound 38 (40 mg) in DMF (0.6 mL) at room temperature, compound 2 (79.7 mg) and DIPEA (50 μL) were added and the resulting mixture was stirred for three days. The reaction mixture was diluted with water and brine and extracted three time with ethyl acetate. The combined organic layers were washed with brine, dried over sodium sulfate, filtered and concentrated to give crude compound 44. This material was used without purification.

Step 2: To a solution of crude compound 44 in acetonitrile (1 mL), aqueous sodium hydroxide (2N, 1 mL) was added followed by a small amount of methanol to give a homogeneous solution. The mixture was stirred at room temperature overnight, then was aqueous HCl (2N, 1 mL) was added to neutralize. Phenol (0.5 g) was added followed by water (2 mL), acetonitrile (1 mL) and concentrated HCl (0.5 mL). The mixture was stirred for 72 hours, then was purified by reverse phase HPLC (Symmetry Shield RPC18, 30×250 mm column, 7 μm, 20-70% methanol in 0.1% trifluoroacetic acid in water, 40 mL/minute). The fraction containing the desired material was lyophilized to give compound 45 as a fluffy off-white solid. The structure was confirmed by LCMS.

### Example 27

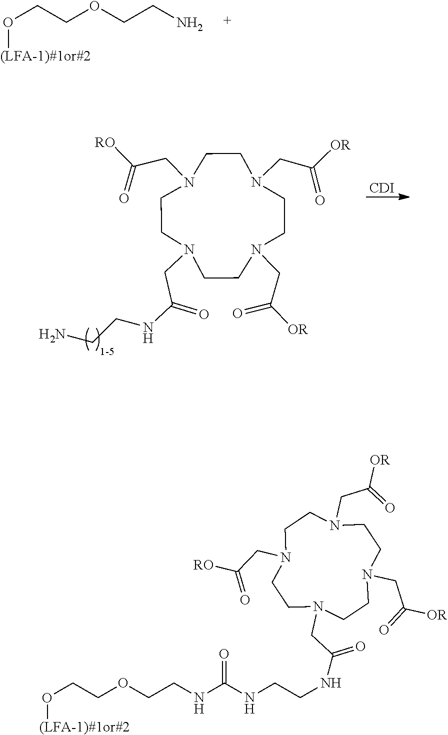

This example shows the synthesis of a LFA-1 antagonist-linker-Tridentate DOTA conjugate.

### Example 28

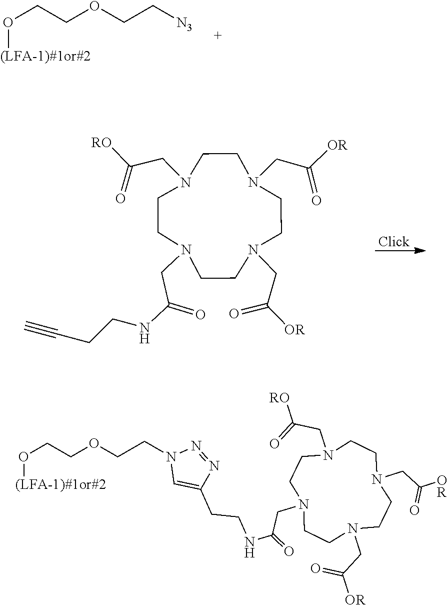

This example shows the synthesis of another LFA-1 antagonist-linker-Tridentate DOTA conjugate.

### Example 29

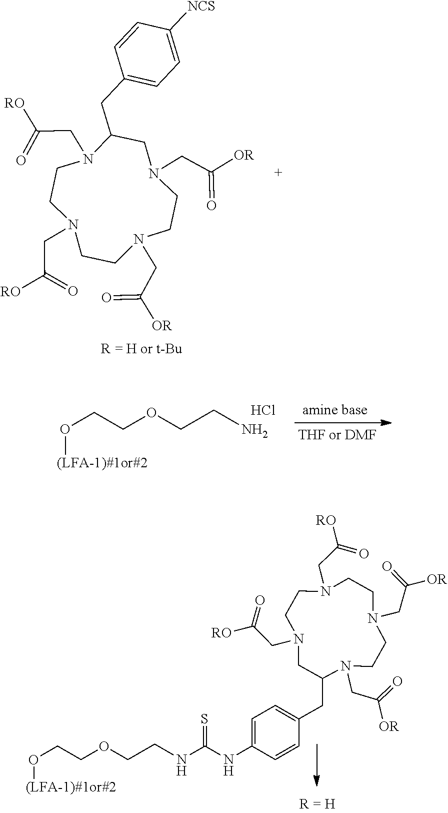

This example shows the synthesis of a LFA-1 antagonist-linker-Tetradentate DOTA conjugate.

### Example 30

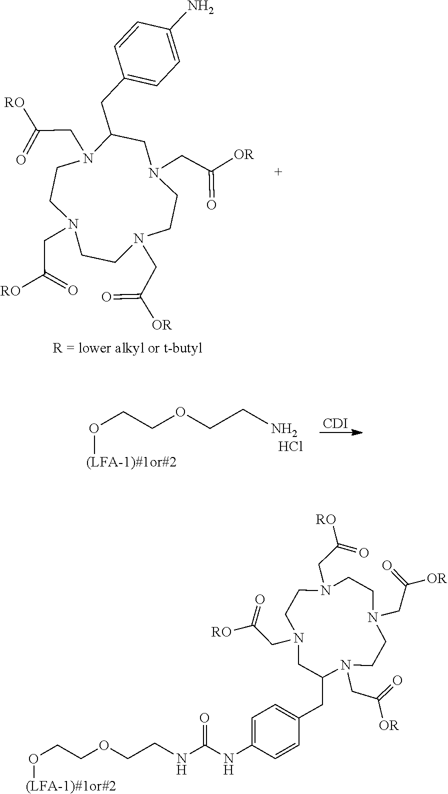

This example shows the synthesis of another LFA-1 antagonist-linker-Tetradentate DOTA conjugate: (tetra-tert-butyl 2,2′,2″,2′″-(2-(4-aminobenzyl)-1,4,7,10-tetraazacyclododecane-1,4,7,10-tetrayl)tetraacetate TFA salt)

### Example 31

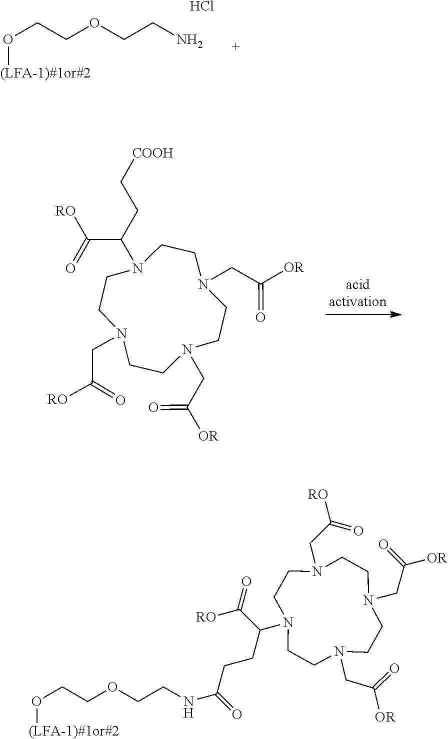

This example shows the synthesis of yet another LFA-1 antagonist-linker-Tetradentate DOTA conjugate.

### Example 32

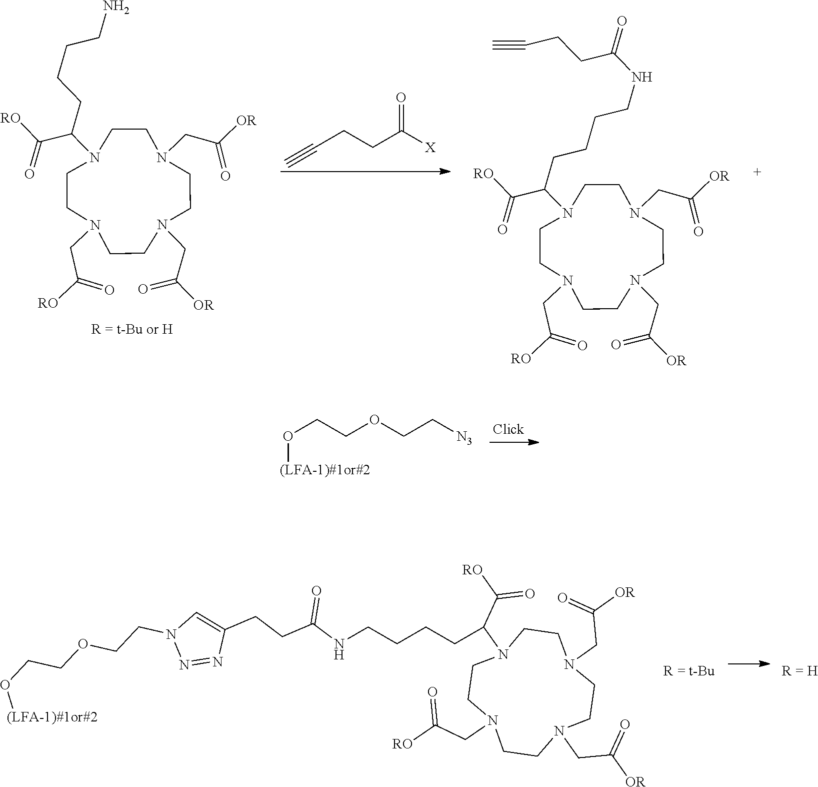

This example shows the synthesis of a LFA-1 antagonist-linker-Tetradentate DOTA conjugate via Click chemistry.

### Example 33

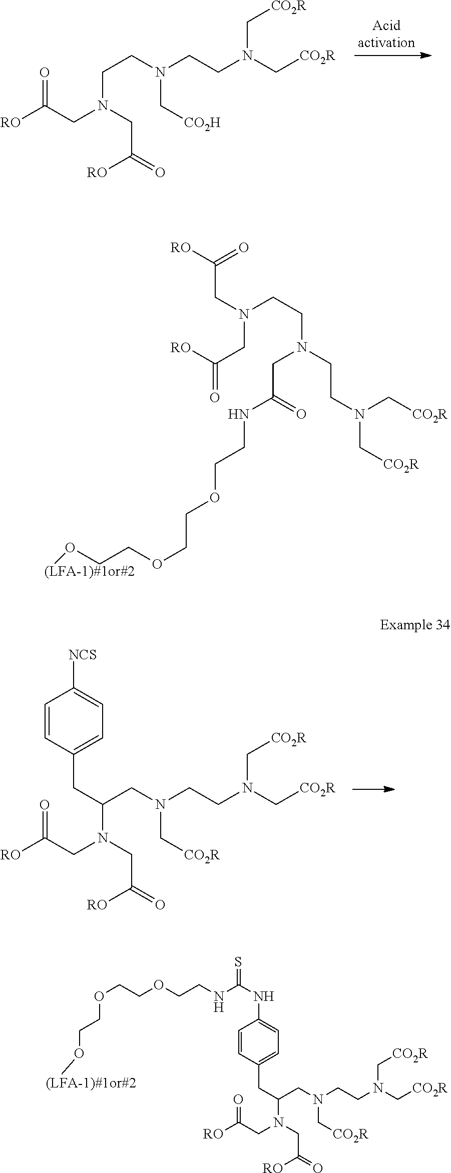

This example shows the synthesis of a LFA-1 antagonist-linker-Pentadentate Ligand conjugate.

### Example 35

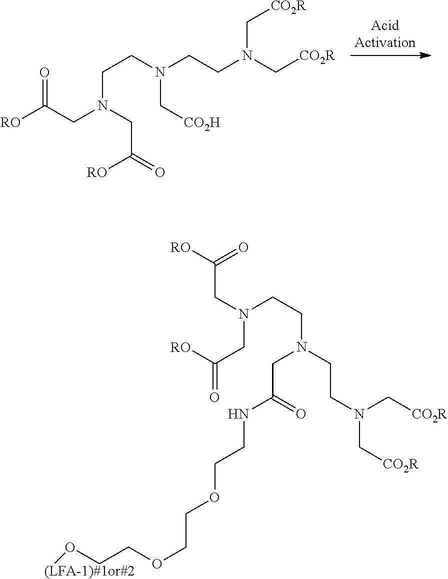

This example shows the synthesis of another LFA-1 antagonist-linker-Pentadentate Ligand conjugate.

### LFA-1 Antagonist-Linker-Dye Conjugates

One skilled in the art could recognize that the same amine based intermediates would follow the same chemistry to install these dyes. Linker may be extended or shortened and include water solubilizing groups.

An example of the LFA-1 antagonist-linked dyes is THI-531-Sulfo-Cy 5. Other dyes include sulfo-Cy5.5 and IR800CW.

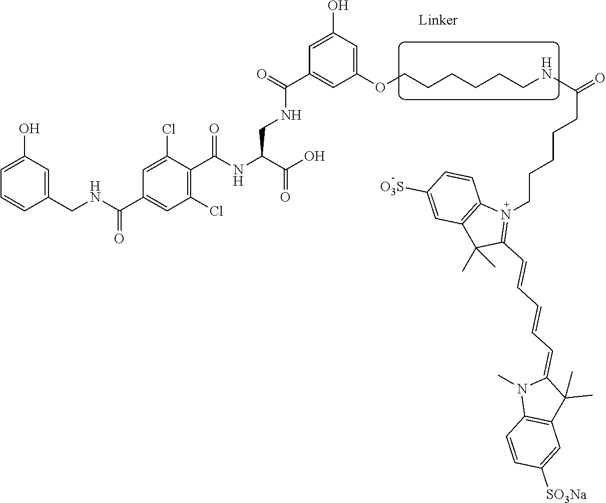

### Example 36

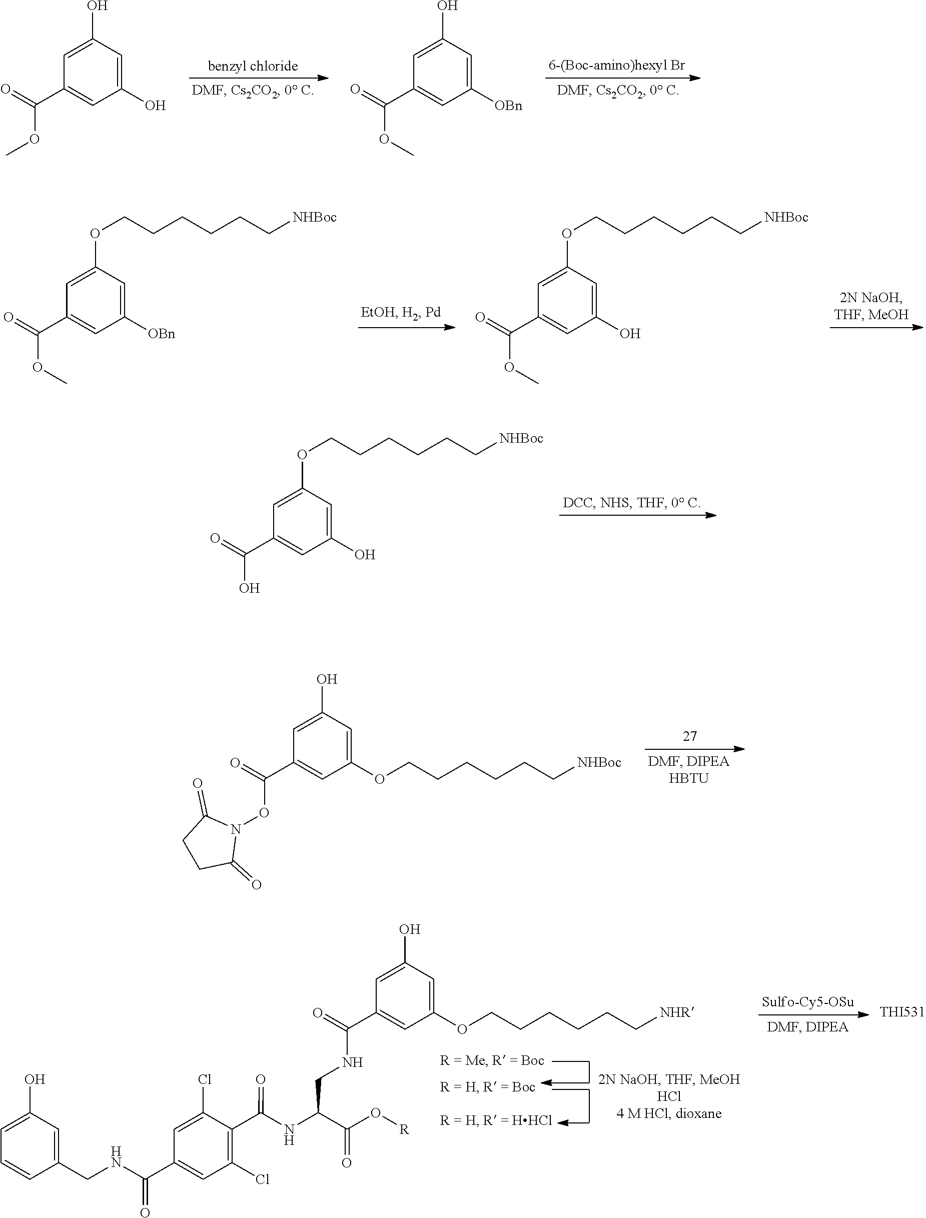

This example shows a representative synthesis of THI531. Stereoisomers may alternatively be synthetized.

While preferred embodiments of the invention have been shown and described, modifications thereof can be made by one skilled in the art without departing from the spirit and teachings of the invention. The embodiments described herein are some only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The disclosures of all patents, patent applications, and publications cited herein are hereby incorporated by reference, to the extent they provide some, procedural or other details supplementary to those set forth herein.

1. A composition comprising a conjugate suitable for imaging, wherein the conjugate formula is integrin targeting component-linker-metal chelating component, wherein the integrin targeting component is a radical derived from a formula selected from a group consisting of 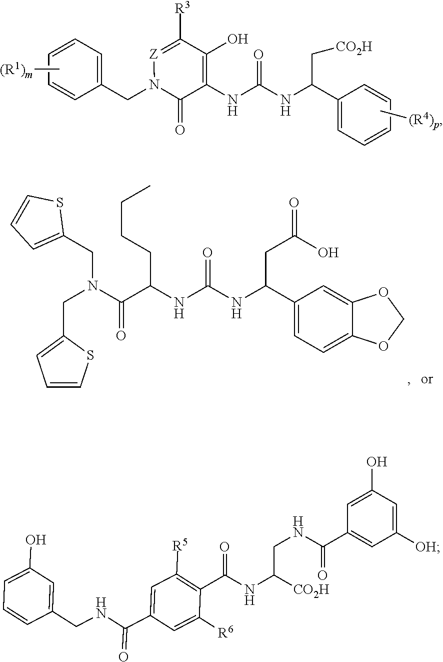
wherein R¹, when present, at each occurrence, is independently selected from the group consisting of halogen, lower alkyl, lower alkenyl, alkynyl, alkoxy, alkenoxy, alkynoxy, thioalkoxy, hydroxyalkyl, aliphatic acyl, —CF₃, —CO₂H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃ alkyl), —NHC(O)NH(C₁-C₆ alkyl), —NHSO₂(C₁-C₃ alkyl), —NHSO₂(aryl), —N(C₁-C₃ alkyl)SO₂(C₁-C₃ alkyl), —N(C₁-C₃ alkyl)SO₂(aryl), alkoxyalkyl, alkylamino, alkenyl amino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl and —C(O)NH(benzyl) groups;
wherein Z is N or C—R²;
wherein R² and R³, when present, are each independently selected from the group consisting of hydrogen, halogen, lower alkyl, lower alkenyl, alkynyl, alkoxy, alkenoxy, alkynoxy, thioalkoxy, hydroxyalkyl, aliphatic acyl, —CF₃, —CO2H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl), —C(O)NH(C₁-C₃ alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃)amino, —C(O)O—(C₁-C₃)alkyl, —C(O)NH—(C₁-C₃)alkyl, —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃(C₁-C₃ alkyl), sulfonamido, carbamate, aryloxyalkyl and —C(O)NH(benzyl) groups; and wherein R² and R³, when present, may be taken together to form a ring;
wherein R⁴, when present, at each occurrence, is independently selected from the group consisting of halogen, lower alkyl, lower alkenyl, alkynyl, alkoxy, alkenoxy, alkynoxy, thioalkoxy, hydroxyalkyl, aliphatic acyl, —CF₃, —CO2H, —SH, —CN, —NO₂, —NH₂, —OH, alkynylamino, alkoxycarbonyl, heterocycloyl, carboxy, —N(C₁-C₃ alkyl)-C(O)(C₁-C₃ alkyl), —NHC(O)N(C₁-C₃ alkyl)C(O)NH(C₁-C₃ alkyl), —NHC(O)NH(C₁-C₆ alkyl), —NHSO₂(C₁-C₃ alkyl), —NHSO₂(aryl), alkoxyalkyl, alkylamino, alkenylamino, di(C₁-C₃ alkyl)amino, —C(O)O—(C₁-C₃)alkyl, —C(O)NH—(C₁-C₃ alkyl), —C(O)N(C₁-C₃ alkyl)₂, —CH═NOH, —PO₃H₂, —OPO₃H₂, haloalkyl, alkoxyalkoxy, carboxaldehyde, carboxamide, cycloalkyl, cycloalkenyl, cycloalkynyl, cycloalkylalkyl, aryl, aroyl, aryloxy, arylamino, biaryl, thioaryl, diarylamino, heterocyclyl, alkylaryl, aralkenyl, aralkyl, alkylheterocyclyl, heterocyclylalkyl, sulfonyl, —SO₂—(C₁-C₃ alkyl), —SO₃—(C₁-C₃ alkyl), sulfonamido, carbamate, aryloxyalkyl, O(haloalkyl), O(cycloalkyl), O(cycloalkylalkyl), piperidinyl, pyrrolidinyl and —C(O)NH(benzyl) groups; and
wherein R¹, R², R³ and R⁴, when present, are each independently unsubstituted or substituted with at least one electron donating or electron withdrawing group;
wherein m and p are independently an integer from 0 to 5;
wherein R⁵ and R⁶ are independently selected from the group of hydrogen, alkyl or halogen;
wherein the linker includes a linear chain having one end attached to the integrin targeting component and another end attached to the metal chelating component, the linear chain consisting of at least 2 atoms and no more than 20 atoms, wherein two or more atoms of the linear chain together with their optional substituents may form a heterocyclic or aryl ring; and
wherein the metal chelating component is a moiety including multiple carboxylic acid groups.

2. The composition of claim 1, wherein the conjugate formula is selected from the group of formulas: 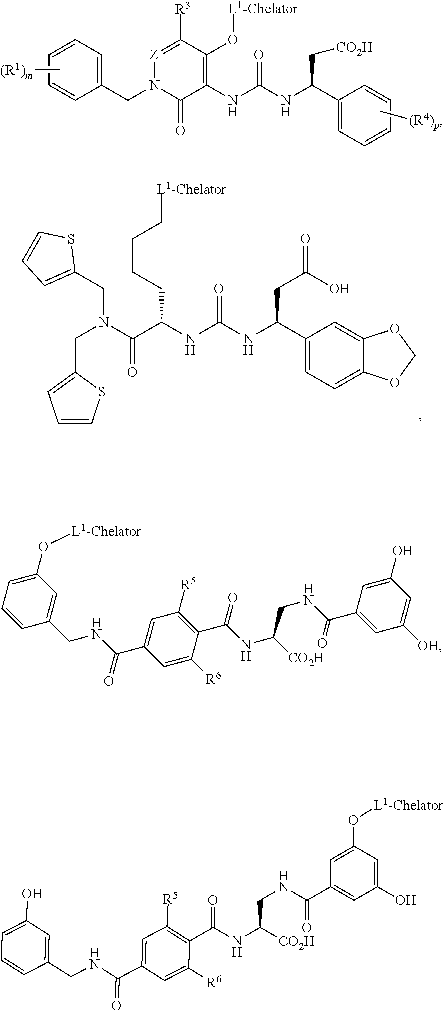
wherein L¹ is said linker, wherein L¹ consists of any combination of one or more of the optionally substituted chemical groups selected from —CH₂—, —CH═CH—, —C(O)—, —NH—, —S—, —S(O)—, —O—, —C(O)O—, —S(O)₂—, a portion of an aryl ring, and a portion of a heterocyclic ring; and
wherein Chelator is said metal chelating component, wherein Chelator consists of a group containing 3 to 5 carboxylic acid functional groups capable of binding to a metal ion.

3. The composition of claim 2, wherein the integrin targeting component is a VLA-4 antagonist, and wherein the conjugate formula is:

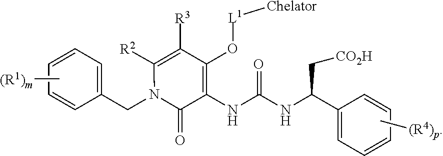

4. The compound of claim 3 wherein:
R² is hydrogen or methyl;
R³ is hydrogen or methyl; and
R¹ and R⁴ are each independently selected from the group consisting of hydrogen, halogen, alkyl, alkoxy, alkoxyalkoxy, hydroxy, and hydroxyalkoxy.

5. The composition of claim 2, wherein the integrin targeting component is a LFA-1 antagonist, and wherein the conjugate formula is:

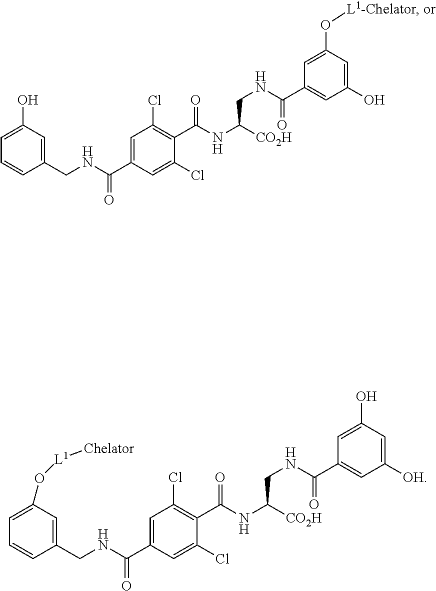

6. The composition of claim 1, wherein the metal chelating component is:
a DOTA derivative (2-[4,7,10-tris(carboxymethyl)-1,4,7,10-tetrazacyclododec-1-yl]acetic acid), or a DTPA (diethylenetriamine pentaacetic acid) derivative, or
a PCTA (3,6,9,15-tetraazabicyclo[9.3.1] pentadeca-1(15),11,13-triene-3,6,9-triacetic acid) derivative.

7. The composition of any of claim 6, further comprising an ion selected from Tm, Gd, Eu, Ho, Cu, Sn, Tc, In and radioisotopes thereof, wherein the conjugate is complexed with the ion.

8. The composition of claim 1, further comprising an ion selected from Tm, Gd, Eu, Ho, Cu, Sn, Tc, In and radioisotopes thereof, wherein the conjugate is complexed with the ion.

9. A pharmaceutical composition containing an effective amount of the composition of claim 8, or a pharmaceutically acceptable salt thereof, in a pharmaceutically acceptable carrier.

10. A method of using the composition of claim 9 in
MRI and/or PET imaging of vulnerable plaques in atherosclerosis; or
MRI and/or PET imaging of lung inflammation in acute lung injury; or
MRI and/or PET imaging of inflamed joints; or
MRI and/or PET imaging of tumors for diagnostic purposes, or purposes of validating therapeutic treatments; or
MRI and/or PET imaging of transplant rejection; or
MRI and/or PET imaging of aortic dissection/aneurysm.

11. The method of claim 10, wherein inflamed joints comprise rheumatoid arthritic joints.

12. A compound, including pharmaceutically acceptable salts, selected from the group consisting of:
(S)-2,2′,2″-(10-(2-((2-(2-(2-((3-(3-(2-carboxy-1-(3-(2-hydroxy ethoxy)phenyl)ethyl)ureido)-1-(2-chloro-6-(2-hydroxyethoxy)benzyl)-5-methyl-2-oxo-1,2-dihydropyridin-4-yl)oxy)ethoxy)ethoxy)ethyl)amino)-2-oxoethyl)-1,4,7,10-tetraazacyclododecane-1,4,7-triyl)triacetic acid, 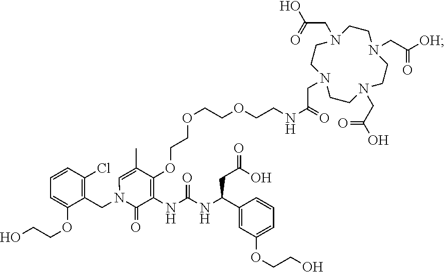 [SMILES: Cc1cn(Cc2c(Cl)cccc2OCCO)c(=O)c(NC(=O)N[C@@H](CC(=O)O)c2cccc(OCCO)c2)c1OCCOCCOCCNC(=O)CN1CCN(CC(=O)O)CCN(CC(=O)O)CCN(CC(=O)O)CC1]
2,2′,2″-(10-(1-carboxy-4-((2-(2-(2-((3-(3-((S)-2-carboxy-1-(3-(2-hydroxyethoxy)phenyl)ethyl)ureido)-1-(2-chloro-6-(2-hydroxyethoxy)benzyl)-5-methyl-2-oxo-1,2-dihydropyridin-4-yl)oxy)ethoxy)ethoxy)ethyl)amino)-4-oxobutyl)-1,4,7,10-tetraazacyclododecane-1,4,7-triyl)triacetic acid, 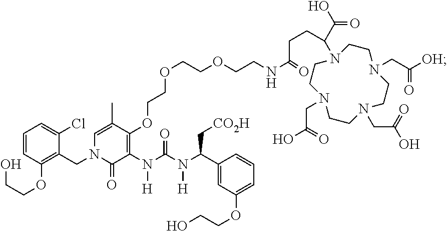 [SMILES: Cc1cn(Cc2c(Cl)cccc2OCCO)c(=O)c(NC(=O)N[C@@H](CC(=O)O)c2cccc(OCCO)c2)c1OCCOCCOCCNC(=O)CCC(C(=O)O)N1CCN(CC(=O)O)CCN(CC(=O)O)CCN(CC(=O)O)CC1]
2,2′,2″,2′″-(2-(4-(3-(2-(2-(2-((3-(3-((S)-2-carboxy-1-(3-(2-hydroxyethoxy)phenyl)ethyl)ureido)-1-(2-chloro-6-(2-hydroxyethoxy)benzyl)-5-methyl-2-oxo-1,2-dihydropyridin-4-yl)oxy)ethoxy)ethoxy)ethyl)ureido)benzyl)-1,4,7,10-tetraazacyclododecane-1,4,7,10-tetrayl)tetraacetic acid, 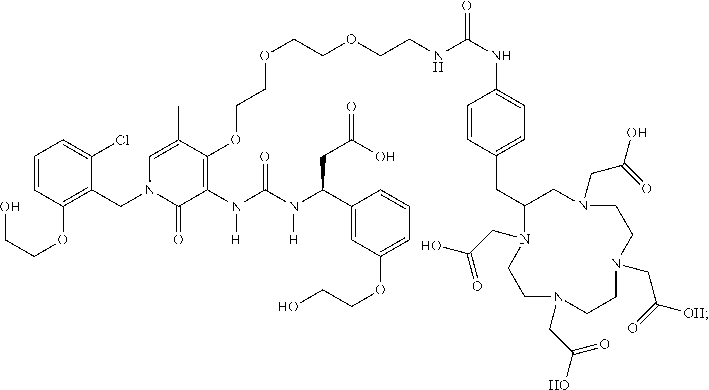 [SMILES: Cc1cn(Cc2c(Cl)cccc2OCCO)c(=O)c(NC(=O)N[C@@H](CC(=O)O)c2cccc(OCCO)c2)c1OCCOCCOCCNC(=O)Nc1ccc(CC2CN(CC(=O)O)CCN(CC(=O)O)CCN(CC(=O)O)CCN2CC(=O)O)cc1]
2,2′,2″,2′″-(2-(4-(3-(2-(2-(2-((3-(3-((S)-2-carboxy-1-(3-(2-hydroxyethoxy)phenyl)ethyl)ureido)-1-(2-chloro-6-(2-hydroxyethoxy)benzyl)-5-methyl-2-oxo-1,2-dihydropyridin-4-yl)oxy)ethoxy)ethoxy)ethyl)thioureido)benzyl)-1,4,7,10-tetraazacyclododecane-1,4,7,10-tetrayl)tetraacetic acid, 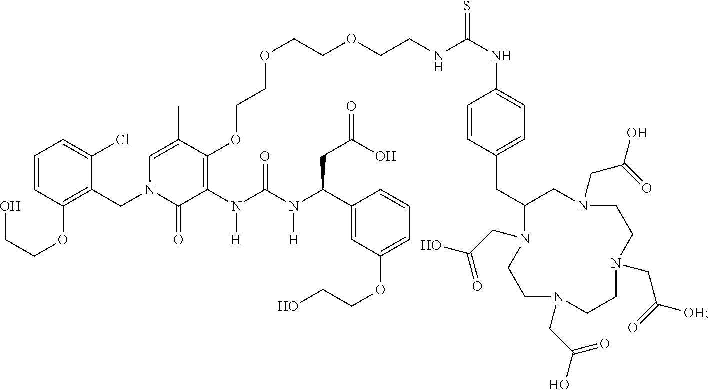 [SMILES: Cc1cn(Cc2c(Cl)cccc2OCCO)c(=O)c(NC(=O)N[C@@H](CC(=O)O)c2cccc(OCCO)c2)c1OCCOCCOCCNC(=S)Nc1ccc(CC2CN(CC(=O)O)CCN(CC(=O)O)CCN(CC(=O)O)CCN2CC(=O)O)cc1]
(S)-2,2′,2″-(10-(2-((2-(2-(3-((2-carboxy-2-(2,6-dichloro-4-((3-hydroxybenzyl)carbamoyl)benzamido)ethyl)carbamoyl)-5-hydroxyphenoxy)ethoxy)ethyl)amino)-2-oxoethyl)-1,4,7,10-tetraazacyclododecane-1,4,7-triyl)triacetic acid, 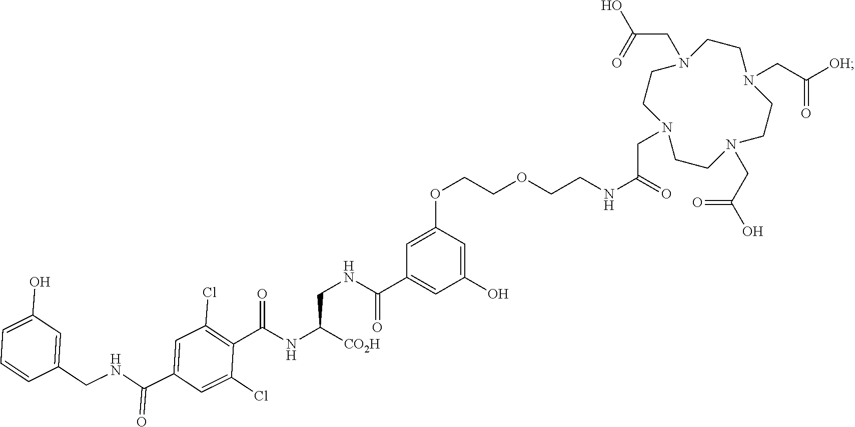 [SMILES: O=C(O)CN1CCN(CC(=O)O)CCN(CC(=O)NCCOCCOc2cc(O)cc(C(=O)NC[C@H](NC(=O)c3c(Cl)cc(C(=O)NCc4cccc(O)c4)cc3Cl)C(=O)O)c2)CCN(CC(=O)O)CC1]
2,2′,2″,2′″-(2-(4-(3-(2-(3-(3-(((S)-2-carboxy-2-(2,6-dichloro-4-((3-hydroxybenzyl)carbamoyl)benzamido)ethyl)carbamoyl)-5-hydroxyphenoxy)propoxy)ethyl)ureido)benzyl)-1,4,7,10-tetraazacyclododecane-1,4,7,10-tetrayl)tetraacetic acid, 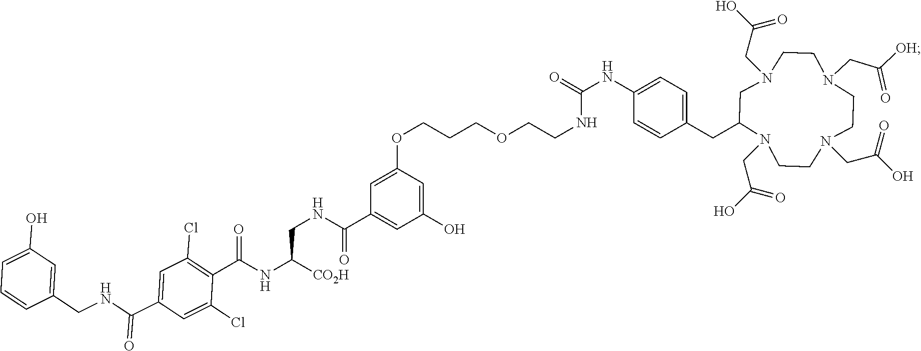 [SMILES: O=C(O)CN1CCN(CC(=O)O)CCN(CC(=O)O)C(Cc2ccc(NC(=O)NCCOCCCOc3cc(O)cc(C(=O)NC[C@H](NC(=O)c4c(Cl)cc(C(=O)NCc5cccc(O)c5)cc4Cl)C(=O)O)c3)cc2)CN(CC(=O)O)CC1]
2,2′,2″,2′″-(2-(4-(3-(2-(2-(3-(((S)-2-carboxy-2-(2,6-dichloro-4-((3-hydroxybenzyl)carbamoyl)benzamido)ethyl)carbamoyl)-5-hydroxyphenoxy)ethoxy)ethyl)thioureido)benzyl)-1,4,7,10-tetraazacyclododecane-1,4,7,10-tetrayl)tetraacetic acid, 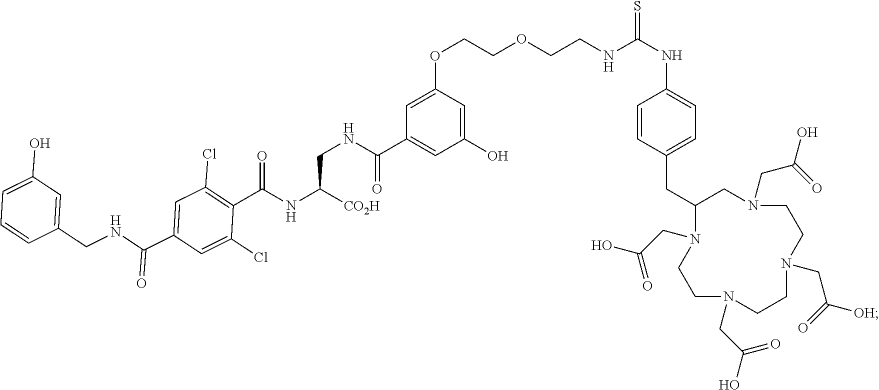 [SMILES: O=C(O)CN1CCN(CC(=O)O)CCN(CC(=O)O)C(Cc2ccc(NC(=S)NCCOCCOc3cc(O)cc(C(=O)NC[C@H](NC(=O)c4c(Cl)cc(C(=O)NCc5cccc(O)c5)cc4Cl)C(=O)O)c3)cc2)CN(CC(=O)O)CC1]
2,2′,2″-(10-(1-carboxy-4-((2-(2-(3-(((S)-2-carboxy-2-(2,6-dichloro-4-((3-hydroxybenzyl)carbamoyl)benzamido)ethyl)carbamoyl)-5-hydroxyphenoxy)ethoxy)ethyl)amino)-4-oxobutyl)-1,4,7,10-tetraazacyclododecane-1,4,7-triyl)triacetic acid, 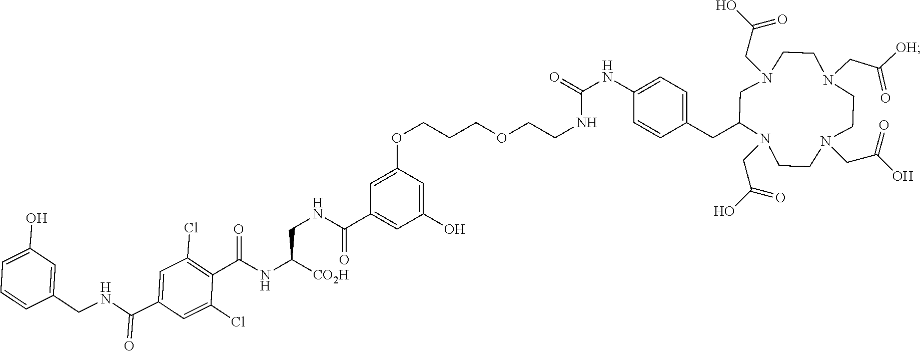 [SMILES: O=C(O)CN1CCN(CC(=O)O)CCN(CC(=O)O)C(Cc2ccc(NC(=O)NCCOCCCOc3cc(O)cc(C(=O)NC[C@H](NC(=O)c4c(Cl)cc(C(=O)NCc5cccc(O)c5)cc4Cl)C(=O)O)c3)cc2)CN(CC(=O)O)CC1]
2,2′,2″,2′″-(2-(4-(3-(2-(2-(3-((4-(((S)-1-carboxy-2-(3,5-dihydroxybenzamido)ethyl)carbamoyl)-3,5-dichlorobenzamido)methyl)phenoxy)ethoxy)ethyl)ureido)benzyl)-1,4,7,10-tetraazacyclododecane-1,4,7,10-tetrayl)tetraacetic acid, 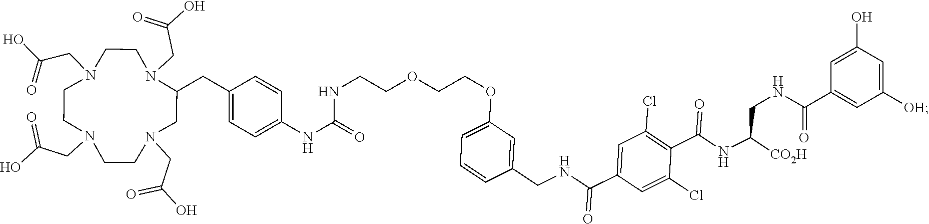 [SMILES: O=C(O)CN1CCN(CC(=O)O)CCN(CC(=O)O)C(Cc2ccc(NC(=O)NCCOCCOc3cccc(CNC(=O)c4cc(Cl)c(C(=O)N[C@@H](CNC(=O)c5cc(O)cc(O)c5)C(=O)O)c(Cl)c4)c3)cc2)CN(CC(=O)O)CC1]
2,2′,2″,2′″-(2-(4-(3-(2-(2-(3-((4-(((S)-1-carboxy-2-(3,5-dihydroxybenzamido)ethyl)carbamoyl)-3,5-dichlorobenzamido)methyl)phenoxy)ethoxy)ethyl)thioureido)benzyl)-1,4,7,10-tetraazacyclododecane-1,4,7,10-tetrayl)tetraacetic acid, 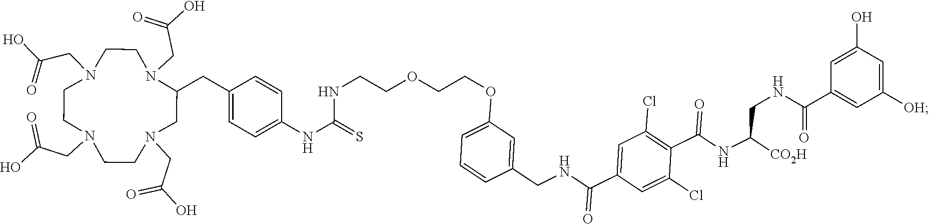 [SMILES: O=C(O)CN1CCN(CC(=O)O)CCN(CC(=O)O)C(Cc2ccc(NC(=S)NCCOCCOc3cccc(CNC(=O)c4cc(Cl)c(C(=O)N[C@@H](CNC(=O)c5cc(O)cc(O)c5)C(=O)O)c(Cl)c4)c3)cc2)CN(CC(=O)O)CC1]
2,2′,2″-(10-(1-carboxy-4-((2-(2-(3-((4-(((S)-1-carboxy-2-(3,5-dihydroxybenzamido)ethyl)carbamoyl)-3,5-dichlorobenzamido)methyl)phenoxy)ethoxy)ethyl)amino)-4-oxobutyl)-1,4,7,10-tetraazacyclododecane-1,4,7-triyl)triacetic acid, 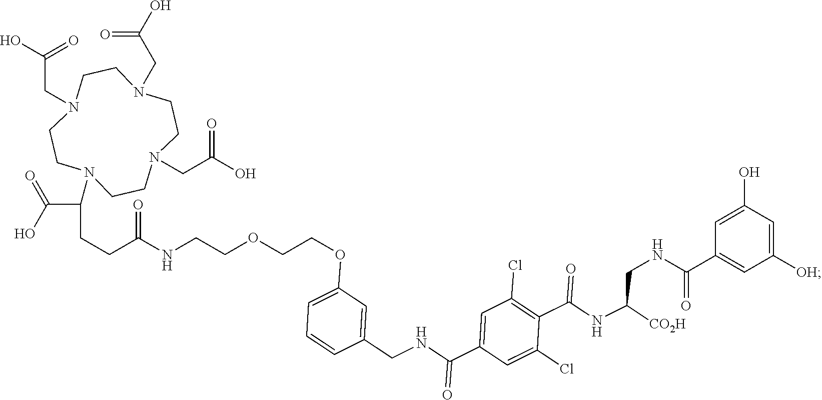 [SMILES: O=C(O)CN1CCN(CC(=O)O)CCN(C(CCC(=O)NCCOCCOc2cccc(CNC(=O)c3cc(Cl)c(C(=O)N[C@@H](CNC(=O)c4cc(O)cc(O)c4)C(=O)O)c(Cl)c3)c2)C(=O)O)CCN(CC(=O)O)CC1]
(S)-2,2′,2″-(10-(2-((2-(2-(2-((3-(3-(2-carboxy-1-(3-(2-ethoxy)phenyl)ethyl)ureido)-1-(2-chloro-6-(2-ethoxy)benzyl)-5-methyl-2-oxo-1,2-dihydropyridin-4-yl)oxy)ethoxy)ethoxy)ethyl)amino)-2-oxoethyl)-1,4,7,10-tetraazacyclododecane-1,4,7-triyl)triacetic acid; 2,2′,2″-(10-(1-carboxy-4-((2-(2-(2-((3-(3-((S)-2-carboxy-1-(3-(2-ethoxy)phenyl)ethyl)ureido)-1-(2-chloro-6-(2-ethoxy)benzyl)-5-methyl-2-oxo-1,2-dihydropyridin-4-yl)oxy)ethoxy)ethoxy)ethyl)amino)-4-oxobutyl)-1,4,7,10-tetraazacyclododecane-1,4,7-triyl)triacetic acid;
2,2′,2″-(10-(1-carboxy-4-((2-(2-(2-((3-(3-((S)-2-carboxy-1-(3-(2-ethoxy)phenyl)ethyl)ureido)-1-(2-ethoxybenzyl)-5-methyl-2-oxo-1,2-dihydropyridin-4-yl)oxy)ethoxy)ethoxy)ethyl)amino)-4-oxobutyl)-1,4,7,10-tetraazacyclododecane-1,4,7-triyl)triacetic acid;
(S)-2,2′,2″-(10-(2-((2-(2-(2-((3-(3-(2-carboxy-1-(3-(2-ethoxy)phenyl)ethyl)ureido)-1-(2-ethoxybenzyl)-5-methyl-2-oxo-1,2-dihydropyridin-4-yl)oxy)ethoxy)ethoxy)ethyl)amino)-2-oxoethyl)-1,4,7,10-tetraazacyclododecane-1,4,7-triyl)triacetic acid; 2,2′,2″,2′″-(2-(4-(3-(2-(2-(2-((3-(3-((S)-2-carboxy-1-(3-(2-ethoxy)phenyl)ethyl)ureido)-1-(2-chloro-6-(2-ethoxy)benzyl)-5-methyl-2-oxo-1,2-dihydropyridin-4-yl)oxy)ethoxy)ethoxy)ethyl)ureido)benzyl)-1,4,7,10-tetraazacyclododecane-1,4,7,10-tetrayl)tetraacetic acid;
2,2′,2″,2′″-(2-(4-(3-(2-(2-(2-((3-(3-((S)-2-carboxy-1-(3-(2-ethoxy)phenyl)ethyl)ureido)-1-(2-ethoxybenzyl)-5-methyl-2-oxo-1,2-dihydropyridin-4-yl)oxy)ethoxy)ethoxy)ethyl)ureido)benzyl)-1,4,7,10-tetraazacyclododecane-1,4,7,10-tetrayl)tetraacetic acid;
2,2′,2″,2′″-(2-(4-(3-(2-(2-(2-((3-(3-((S)-2-carboxy-1-(3-(2-ethoxy)phenyl)ethyl)ureido)-1-(2-chloro-6-(2-ethoxy)benzyl)-5-methyl-2-oxo-1,2-dihydropyridin-4-yl)oxy)ethoxy)ethoxy)ethyl)thioureido)benzyl)-1,4,7,10-tetraazacyclododecane-1,4,7,10-tetrayl)tetraacetic acid;
2,2′,2″,2′″-(2-(4-(3-(2-(2-(2-((3-(3-((S)-2-carboxy-1-(3-(2-ethoxy)phenyl)ethyl)ureido)-1-(2-ethoxybenzyl)-5-methyl-2-oxo-1,2-dihydropyridin-4-yl)oxy)ethoxy)ethoxy)ethyl)thioureido)benzyl)-1,4,7,10-tetraazacyclododecane-1,4,7,10-tetrayl)tetraacetic acid.

13. A compound of claim 12 further comprising an ion selected from Tm, Gd, Eu, Ho, Cu, Sn, Tc, In and radioisotopes thereof, wherein the conjugate is complexed with the ion.

14. A method of using a composition comprising a conjugate of the formula VLA-4 antagonist-linker-chelator or LFA-1 antagonist-linker-chelator in anti-inflammatory or immunosuppressive drug delivery in atherosclerosis; or
delivery of immunosuppressive therapeutics to immune cells to prevent acute or chronic transplant rejection; or
delivery of immunosuppressive therapeutics to immune cells in autoimmune diseases; or
delivery of therapeutic agents to tumors or malignant cells.

15. The method of claim 14, wherein autoimmune diseases comprise multiple sclerosis or systemic lupus erythematosus.
